(19) United States
(12) Patent Application Publication
(10) Pub. No.: US 2016/0213689 A1
(43) Pub. Date: Jul. 28, 2016

(54) Treatment of Pulmonary Disease

(71) Applicant: Intercept Pharmaceuticals, Inc., New York, NY (US)

(72) Inventors: Mark Pruzanski, New York, NY (US); Luciano Adorini, Milano (IT)

(21) Appl. No.: 15/008,665

(22) Filed: Jan. 28, 2016

Related U.S. Application Data
(63) Continuation of application No. 14/090,815, filed on Nov. 26, 2013, now abandoned.
(60) Provisional application No. 61/730,749, filed on Nov. 28, 2012.

(51) Int. Cl. A61K 31/575

(52) U.S. Cl. CPC A61K 31/575

(57) ABSTRACT

The present invention relates to methods of treating, reducing the risk of, preventing, or alleviating a symptom of a pulmonary disease or condition, reducing or suppressing inflammation in the lung, and promoting lung repair, by using a compound of formula A:

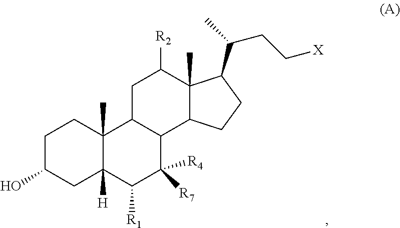

or a pharmaceutically acceptable salt thereof.

### CROSS-REFERENCE TO RELATED APPLICATION

This application claims priority to, and the benefit of, U.S. Application No. 61/730,749, filed on Nov. 28, 2012, the entire contents of which are incorporated herein by reference.

### BACKGROUND OF THE INVENTION

Pulmonary diseases, commonly known as lung diseases, represent the third leading cause of death in the US. The most frequently diagnosed pulmonary diseases include emphysema, asthma, pneumonia, tuberculosis, pulmonary hypertension, and lung cancer. Pulmonary hypertension is a chronic and progressive disease. The key pathologic change in pulmonary hypertension is the remodeling of small pulmonary arteries, characterized by thickening of the intima, media, and adventitia. The progressive narrowing of the pulmonary microvascular bed, and the subsequent increase in vascular resistance, reduce their capacity to carry blood and causes an increase in pressure. Over time, the increased pressure induces an adaptive hypertrophy in the right ventricle (RV) and eventually causes heart failure and leads to patient death.

Pulmonary hypertension may be caused by a combination of factors including autoimmune diseases such as scleroderma and rheumatoid arthritis, birth defects of the heart, blood clots in the lungs (pulmonary embolism), congestive heart failure, heart valve disease, HIV infection, extended periods of low oxygen levels in the blood, lung disease such as COPD and pulmonary fibrosis, various medications and substances of abuse, and/or obstructive sleep apnea. Although the exact pathophysiology of pulmonary hypertension remains unknown, there is increasing evidence for a crucial role of inflammation and activation of the innate and adaptive immunities in the development and progression of pulmonary hypertension (Price et al., Chest 2012, 141:210-221).

Several therapeutic agents have been developed for the medical management of pulmonary hypertension, including prostanoids, endothelin receptor antagonists, phosphodiesterase type 5 inhibitors, soluble guanylate cyclase stimulators, and two PDE5 inhibitors, tadalafil and sildenafil. Nitric oxide (NO) is a potent relaxant agent for smooth muscle cells in the pulmonary arteries, exerting its activity through cyclic GMP (cGMP). Intracellular cGMP level depends on the activation of a number of phosphodiesterase (PDEs), among which PDE5 is the most abundantly expressed isoform in the pulmonary circulation.

Acute Lung Injury (ALI) and its more severe form, Acute Respiratory Distress Syndrome (ARDS), are characterized by an acute inflammatory response localized to the air spaces and lung parenchyma of the lungs. ALI and ARDS are major causes of acute respiratory failure, and are associated with high morbidity and mortality in critically ill patients. ARDS may account for 36,000 deaths per year in a country the size of the US. Despite advances in ALI and ARDS patient management, such as lung-protective ventilation, there still exists a need for effective treatments.

The farnesoid X receptor (FXR) is a member of the nuclear receptor superfamily that is highly expressed in different organs, including adipose tissue, liver, kidney, adrenals, intestine and vascular bed (Lefebvre, Physiol. Rev. 2009). FXR signaling modulates several metabolic pathways, regulating triglyceride, cholesterol, glucose and energy homeostasis, and potentially affects the pathogenesis of atherosclerosis by increasing NO production and reducing neointima proliferation and vascular inflammation (Lefebvre, Physiol. Rev. 2009). FXR is also expressed in rat pulmonary artery endothelial cells (ECs) (He, F., et al., Circulation Research 2006, 98: 192-199). Activation of FXR in ECs leads to downregulation of endothelin (ET)-1 expression, a potent vasoconstrictive substance. Manipulation of ET-1 expression in vascular ECs may be useful in controlling pulmonary hypertension. Also, FXR activation suppresses inflammation in the lungs and promotes lung repair after injury. FXR knock-out mice showed increased inflammation in the lungs and defective lung regeneration after acute lung injury induced by lipopoly-saccharide treatment. In vitro, FXR activation was shown to suppress the expression of P-selectin and induce Foxm 1b expression. Together these effects serve to decrease the permeability of the lung, suppress the movement of leukocytes out of circulation and into inflamed tissues, and promote lung repair in an inflammatory mouse model (Zhang, L., Mol. Endocrinol. 2012, 26(1): 27-36). Similar results were observed in the pulmonary fibrosis mouse model (Zhou et al., 2013, 761-65). These findings support the potential ability of FXR or its agonist to suppress lung injury and promote lung repair for treating inflammation-induced lung injury.

Because current treatments are inefficient to improve survival of patients suffering from pulmonary disease, such as pulmonary hypertension, alternative therapies are urgently needed. The present invention addresses such needs.

### BRIEF DESCRIPTION OF THE DRAWINGS

FIG. 1 is a chart showing the study outline and the schedule to perform blood pressure, urine, and blood analyses on the Dahl salt-sensitive (DSS) rats. The DSS rats include four groups (see Example 4, herein referred to as “DSS study rats”).

FIG. 2 is a graph indicating the body weight (gram) of the DSS study rats versus time (week).

FIG. 3 is a graph indicating the survival rate (%) of DSS study rats versus time (week).

FIG. 4 is a graph indicating the heart rate (bpm) of DSS study rats versus time (week).

FIG. 5 is a graph indicating the systolic blood pressure (SBP; mmHg) of DSS study rats versus time (week).

FIG. 6A is a graph indicating the heart mass % of body weight (BW) of DSS study rats.

FIG. 6B is a graph indicating the lung mass % of BW of DSS study rats.

FIG. 6C is a graph indicating the kidney mass % of BW of DSS study rats.

FIG. 7 is a graph indicating the fasting blood glucose concentration (mg/dL) over time (min) during glucose tolerance test (GTT) in DSS study rats.

FIG. 8 is a graph indicating the fasting plasma insulin concentration (ng/mL) over time (min) during GTT in DSS study rats.

FIG. 9 is a histogram indicating the insulin sensitivity using insulin resistance (IR) index in DSS study rats.

FIG. 10A is a graph indicating the urinary albumin (mg/day) of DSS study rats.

FIG. 10B is a graph indicating the urinary albumin to creatinine ratio (UACR) of DSS study rats.

FIG. 11A is a graph indicating the serum ADMA levels (μmol/L) of DSS study rats over time (week).

FIG. 11B is a graph indicating the urinary ADMA levels (nmol/body) of

DSS study rats over time (week).

FIG. 11C is a graph indicating the serum NO levels (μmol/L) of DSS study rats over time (week).

FIG. 11D is a graph indicating the urinary NO levels (nmol/body) of DSS study rats over time (week).

FIG. 12 is a plot indicating the degree of right ventricular hypertrophy (RVH) for each treatment group of DSS study rats following animal sacrifice.

FIGS. 13A-D are 20× magnified images of hematoxylin-eosin stained lung sections taken from rats in the control group (A), monocrotaline treated group (B), monocrotaline plus obeticholic acid (OCA) treated group (C), and monocrotaline plus tadalafil treated group (D) on day 7. The long arrows indicate vessel lumen, and the short arrows indicate vessel wall.

FIG. 13E is a histogram indicating the pulmonary artery wall thickness on day 7 in the treated rats as compared to that for the control group rats. *p<0.0001 vs. control, ^{o}p<0.0001 vs. monocrotaline, and n: the number of arteries evaluated.

FIGS. 14A-D are 20× magnified images of hematoxylin-eosin stained lung sections taken from rats in the control group (A), monocrotaline treated group (B), monocrotaline plus OCA treated group (C), and monocrotaline plus tadalafil treated group (D) on day 28. The long arrows indicate vessel lumen, and the short arrows indicate vessel wall.

FIG. 14E is a histogram indicating the pulmonary artery wall thickness on day 28 in the treated rats as compared to that for the control group rats. *p<0.0001 vs. control, ^{o}p<0.0001 vs. monocrotaline, and n: the number of arteries evaluated.

FIG. 15 is a plot indicating the effect of OCA on mRNA expression of MCP-1 in the control and test groups on days 7 and 28.

FIG. 16 is a plot indicating the effect of OCA on mRNA expression of IL-6 in the control and test groups on days 7 and 28.

FIG. 17 is a plot indicating the effect of OCA on mRNA expression of VEGF in the control and test groups on days 7 and 28.

FIG. 18 is a plot indicating the effect of OCA on mRNA expression of

ACE2 in the control and test groups on days 7 and 28.

FIG. 19 is a plot indicating the effect of OCA on mRNA expression of PKG1 in the control and test groups on days 7 and 28.

FIG. 20 is a plot indicating the effect of OCA on mRNA expression of GC1a3 in the control and test groups on days 7 and 28.

FIG. 21 is a plot indicating the effect of OCA on mRNA expression of PDE5 in the control and test groups on days 7 and 28.

FIG. 22 is a graph depicting univariate analysis of survival in untreated or treated rats over time (days).

### SUMMARY OF THE INVENTION

The invention relates to a method of treating, reducing the risk of, preventing, or alleviating a symptom of a pulmonary disease or condition in a subject, comprising administering to the subject a therapeutically effective amount of a compound of formula A:

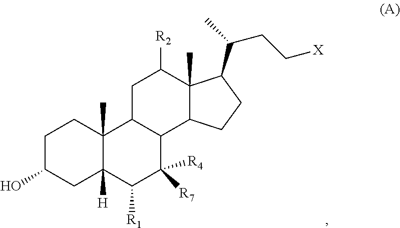

or a pharmaceutically acceptable salt thereof, wherein R₁, R₂, R₄, R₇, and X are as defined herein.

The invention also relates to use of a compound of formula A or a pharmaceutically acceptable salt thereof, in the manufacture of a medicament for the treatment, reducing the risk of, prevention, or alleviation of a symptom of a pulmonary disease or condition in a subject, wherein R₁, R₂, R₄, R₇, and X are as defined herein.

The invention also relates to a compound of formula A or a pharmaceutically acceptable salt thereof, for the treatment, reducing the risk of, prevention, or alleviation of a symptom of a pulmonary disease or condition in a subject, wherein R₁, R₂, R₄, R₇, and X are as defined herein.

The invention further relates to a method of reducing or suppressing inflammation in the lung in a subject, comprising administering to the subject a therapeutically effective amount of a compound of formula A:

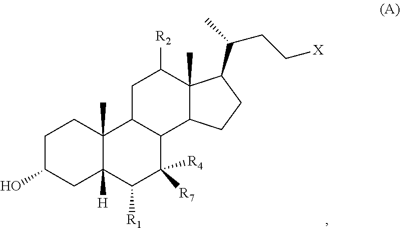

or a pharmaceutically acceptable salt thereof, wherein R₁, R₂, R₄, R₇, and X are as defined herein.

The invention also relates to use of a compound of formula A or a pharmaceutically acceptable salt thereof, in the manufacture of a medicament for reducing or suppressing inflammation in the lung in a subject, wherein R₁, R₂, R₄, R₇, and X are as defined herein.

The invention also relates to a compound of formula A or a pharmaceutically acceptable salt thereof, for reducing or suppressing inflammation in the lung in a subject, wherein R₁, R₂, R₄, R₇, and X are as defined herein.

The invention further relates to a method of promoting lung repair in a subject, comprising administering to the subject a therapeutically effective amount of a compound of formula A:

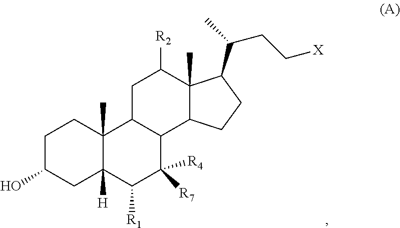

or a pharmaceutically acceptable salt thereof, wherein R₁, R₂, R₄, R₇, and X are as defined herein.

The invention also relates to use of a compound of formula A or a pharmaceutically acceptable salt thereof, in the manufacture of a medicament for promoting lung repair in a subject, wherein R₁, R₂, R₄, R₇, and X are as defined herein.

The invention also relates to a compound of formula A or a pharmaceutically acceptable salt thereof, for promoting lung repair in a subject, wherein R₁, R₂, R₄, R₇, and X are as defined herein.

The invention further relates to a pharmaceutical composition comprising a compound of formula A or a pharmaceutically acceptable salt thereof, and a pharmaceutically acceptable carrier or excipient for the for treating, reducing the risk of, preventing, or alleviating a symptom of a pulmonary disease or condition, or for reducing or suppressing inflammation in the lung, or for promoting lung repair in a subject, wherein R₁, R₂, R₄, R₇, and X are as defined herein.

The invention further relates to a kit comprising a compound of the invention for use in a method of treating, reducing the risk of, preventing, or alleviating a symptom of a pulmonary disease or condition, or of reducing or suppressing inflammation in the lung, or of promoting lung repair in a subject, wherein R₁, R₂, R₄, R₇, and X are as defined herein.

Unless otherwise defined, all technical and scientific terms used herein have the same meaning as commonly understood by one of ordinary skill in the art to which this invention belongs. In the case of conflict, the present specification, including definitions, will control. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference. The references cited herein are not admitted to be prior art to the claimed invention. In addition, the materials, methods, and examples are illustrative only and are not intended to be limiting.

Other features and advantages of the invention will be apparent from the following detailed description and claims.

### DETAILED DESCRIPTION OF THE INVENTION

The invention relates to a method of treating, reducing the risk of, preventing, or alleviating a symptom of a pulmonary disease or condition, a method of reducing or suppressing inflammation in the lung, and a method of promoting lung repair by administering an FXR agonist to a subject in need thereof.

Specifically, the invention relates to a method of treating, reducing the risk of, preventing, or alleviating a symptom of a pulmonary disease or condition, comprising administering to a subject in need thereof a compound of formula A:

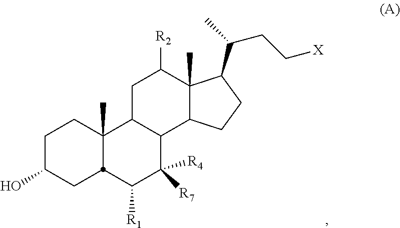

or a pharmaceutically acceptable salt thereof, wherein R₁, R₂, R₄, R₇, and X are as described herein.

The invention also relates to a method of reducing or suppressing inflammation in the lung, comprising administering to a subject in need thereof a compound of formula A or a pharmaceutically acceptable salt thereof, wherein R₁, R₂, R₄, R₇, and X are as described herein.

The invention also relates to a method of promoting lung repair, comprising administering to a subject in need thereof a compound of formula A or a pharmaceutically acceptable salt thereof, wherein R₁, R₂, R₄, R₇, and X are as described herein.

In one embodiment, the methods of the invention comprise administering to a subject in need thereof a compound as described herein. For example, the compound is a compound as described in paragraphs [00067]-[00082].

The invention further relates to use of a compound of formula A:

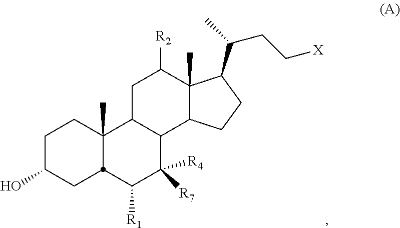

or a pharmaceutically acceptable salt thereof, in the manufacture of a medicament for the treatment, reducing the risk of, prevention, or alleviation of a symptom of a pulmonary disease or condition in a subject, wherein R₁, R₂, R₄, R₇, and X are as defined herein.

The invention also relates to use of a compound of formula A or a pharmaceutically acceptable salt thereof, in the manufacture of a medicament for reducing or suppressing inflammation in the lung in a subject, wherein R₁, R₂, R₄, R₇, and X are as defined herein.

The invention also relates to a compound of formula A or a pharmaceutically acceptable salt thereof, for promoting lung repair in a subject, wherein R₁, R₂, R₄, R₇, and X are as defined herein.

In one embodiment, the compound for the uses of the invention in the manufacture of a medicament is a compound as described herein. For example, the compound is a compound as described in paragraphs [00067]-[00082].

The invention also relates to a compound of formula A:

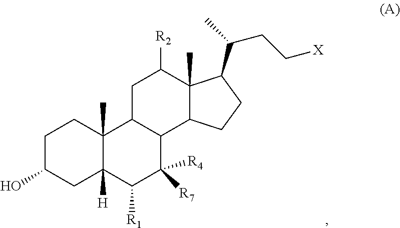

or a pharmaceutically acceptable salt thereof, for the treatment, reducing the risk of, prevention, or alleviation of a symptom of a pulmonary disease or condition in a subject, wherein R₁, R₂, R₄, R₇, and X are as defined herein.

The invention also relates to a compound of formula A or a pharmaceutically acceptable salt thereof, for reducing or suppressing inflammation in the lung in a subject, wherein R₁, R₂, R₄, R₇, and X are as defined herein.

The invention also relates to a compound of formula A or a pharmaceutically acceptable salt thereof, for promoting lung repair in a subject, wherein R₁, R₂, R₄, R₇, and X are as defined herein.

In one embodiment, the compound for the treatment, reducing the risk of, prevention, or alleviation of a symptom of a pulmonary disease or condition, or for reducing or suppressing inflammation in the lung, or for promoting lung repair is a compound as described herein. For example, the compound is a compound as described in paragraphs [00067]-[00082].

The compound used in the invention is a compound of formula A:

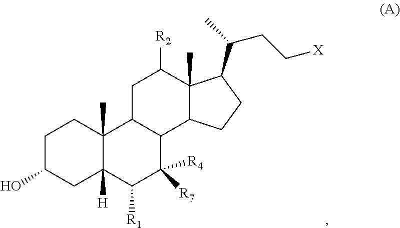

or a pharmaceutically acceptable salt thereof, wherein:
R₁ is hydrogen or unsubstituted C₁-C₆ alkyl;
R₂ is hydrogen or α-hydroxyl;
X is C(O)OH, C(O)NH(CH₂)ₘSO₃H, C(O)NH(CH₂)ₙCO₂H, or OSO₃H;
R₄ is hydroxyl or hydrogen;
R₇ is hydroxyl or hydrogen;
m is 1, 2, or 3; and
n is 1, 2, or 3.

In one embodiment, the compound used in the invention is a salt of a compound of formula A. In one embodiment, the compound used in the invention is a cation salt of a compound of formula A, where the X is converted to the corresponding anion. For example, X is converted to an anion selected from C(O)O⁻, C(O)NH(CH₂)ₘSO₃⁻, C(O)NH(CH₂)ₙCO₂⁻, or OSO₃⁻.

In one embodiment, the compound used in the invention is a sodium salt of a compound of formula A, for example, a compound of formula A wherein X is converted to OSO₃⁻ and forms a salt with Na⁺. In one embodiment, the compound used in the invention is a triethylammonium salt of a compound of formula A, for example, a compound of formula A wherein X is converted to OSO₃⁻ and forms a salt with Et₃NH⁻.

In one embodiment, the compound used in the invention is a compound of formula A, wherein R₁ is unsubstituted C₁-C₆ alkyl. In a further embodiment, the compound used in the invention is a compound of formula A, wherein R₁ is unsubstituted C₁-C₃ alkyl. In a further embodiment, the compound used in the invention is a compound of formula A, wherein R₁ is selected from methyl, ethyl, and propyl. In a further embodiment, the compound used in the invention is a compound of formula A, wherein R₁ is ethyl.

In one embodiment, the compound used in the invention is a compound of formula A, wherein X is selected from C(O)OH, C(O)NH(CH₂)ₘSO₃H, and C(O)NH(CH₂)ₙCO₂H. In a further embodiment, the compound used in the invention is a compound of formula A, wherein X is selected from C(O)OH, C(O)NH(CH₂)SO₃H, C(O)NH(CH₂)CO₂H, C(O)NH(CH₂)₂SO₃H, C(O)NH(CH₂)₂CO₂H. In a further embodiment, the compound used in the invention is a compound of formula A, wherein X is C(O)OH. In another embodiment, the compound used in the invention is a compound of formula A, wherein X is OSO₃H. In another embodiment, the compound used in the invention is a compound of formula A, wherein X is OSO₃⁻Na⁺. In another embodiment, the compound used in the invention is a compound of formula A, wherein X is OSO₃⁻NHEt₃⁺.

In one embodiment, the compound used in the invention is a compound of formula A, wherein R₁ is selected from methyl, ethyl and propyl, R₄ is OH, R₇ is H, and R₂ is H.

In one embodiment, the compound used in the invention is a compound of formula I or IA:

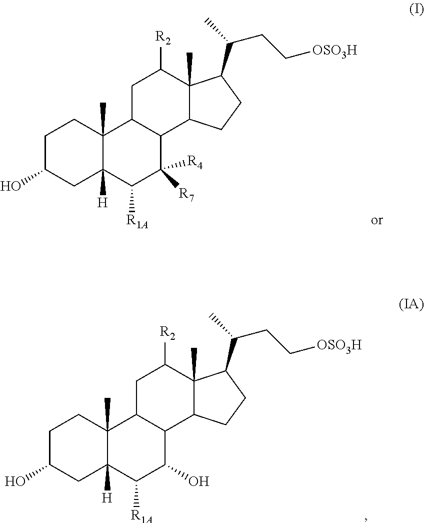

or a pharmaceutically acceptable salt thereof, wherein
R_{1A} is hydrogen or unsubstituted C₁-C₆ alkyl;
R₂ is hydrogen or α-hydroxyl;
R₄ is hydroxyl or hydrogen; and
R₇ is hydroxyl or hydrogen.

In one embodiment, the compound used in the invention is a sodium salt of formula I or IA. In one embodiment, the compound of the invention is a triethylammonium salt of a compound of formula I or IA.

In one embodiment, the compound used in the invention is a compound of formula II or IIA:

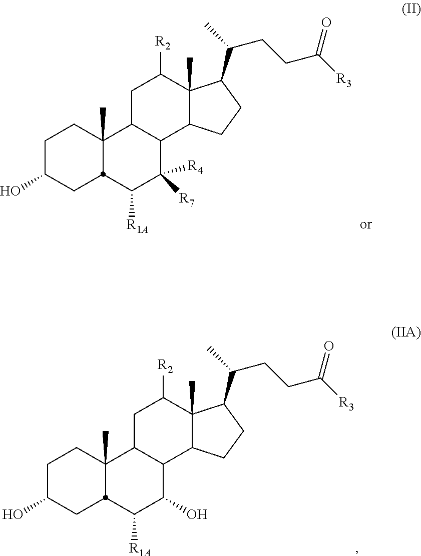

or a pharmaceutically acceptable salt thereof, wherein:
R_{1A} is hydrogen or unsubstituted C₁-C₆ alkyl;
R₂ is hydrogen or α-hydroxyl;
R₃ is hydroxyl, NH(CH₂)ₘSO₃H, or NH(CH₂)ₙCO₂H;
R₄ is hydroxyl or hydrogen; and
R₇ is hydroxyl or hydrogen;

In one embodiment, the compound used in the invention is a compound of formula II or IIA, wherein R₃ is selected from OH, NH(CH₂)SO₃H, NH(CH₂)CO₂H, NH(CH₂)₂SO₃H, and NH(CH₂)₂CO₂H. In a further embodiment, the compound used in the invention is a compound of formulae II or IIA, wherein R₃ is OH.

In one embodiment, the compound used in the invention is a compound of formula A, I, IA, II or IIA, wherein R₂ is hydrogen.

In one embodiment, the compound used in the invention is a compound of formulae A, I, or II, wherein R₄ is hydroxyl and R₇ is hydrogen.

In one embodiment, the compound used in the invention is a compound of formula I, IA, II, or IIA, wherein R_{1A} is unsubstituted C₁-C₆ alkyl. In a further embodiment, the compound used in the invention is a compound of formula I, IA, II, or IIA, wherein R_{1A} is unsubstituted C₁-C₃ alkyl. In a further embodiment, the compound used in the invention is a compound of formula I, IA, II, or IIA, wherein R1A is selected from methyl, ethyl, and propyl. In a further embodiment, the compound used in the invention is a compound of formula I, IA, II, or IIA, wherein R_{1A} is ethyl.

In one embodiment, the compound used in the invention is a compound selected from

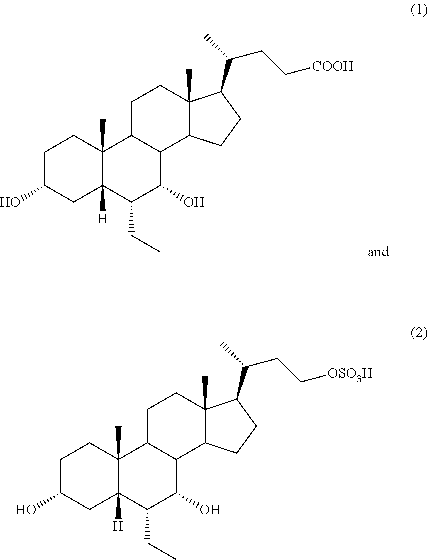

or a pharmaceutically acceptable salt thereof.

Compound 1 is also referred to as 6ECDCA or obeticholic acid (OCA).

In one embodiment, the compound used in the invention is a compound selected from

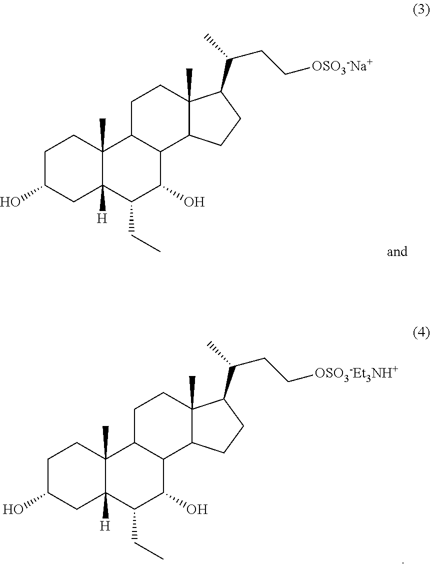

Compounds of formula I, IA, II, or IIA are subsets of compounds of formula A. Features described herein for compounds of formula A apply equally to compounds of formula I, IA, II, or IIA.

The compounds of the invention may be readily prepared by those skilled in the art. In particular, compounds of the invention may be prepared according to the published procedures in U.S. Pat. Nos. 7,786,102, 7,994,352, and/or 7,932,244.

The compounds described herein are suitable for the treatment, reducing the risk of, prevention, or alleviation of a symptom of a variety of pulmonary diseases or conditions. Pulmonary diseases and conditions are considered to be those that affect the pulmonary or lung system in the body. Without the intention of being bound by the theory, compounds of the invention are suitable for the treatment, reducing the risk of, prevention, or alleviation of a symptom of a variety of pulmonary diseases or conditions by increasing NO production, downregulating endothelin (ET)-1, decreasing the permeability of the lung, and/or suppressing the movement of leukocytes or fibrocytes from circulation to inflamed tissues.

In one embodiment, the compounds described herein are suitable for the treatment, reducing the risk of, prevention, or alleviation of a symptom of pulmonary diseases or conditions caused by or associated with inflammation, autoimmune diseases such as scleroderma and rheumatoid arthritis, Acute Lung Injury (ALI), Acute Respiratory Distress Syndrome (ARDS), birth defects of the heart, blood clots in the lungs (pulmonary embolism), congestive heart failure, heart valve disease, HIV infection, extended periods of low oxygen levels in the blood, various medications and substances of abuse, and/or obstructive sleep apnea. In one embodiment, the pulmonary diseases or conditions are caused by or associated with inflammation of the lungs. In another embodiment, the pulmonary diseases or conditions are caused by or associated with ALI or ARDS.

In one embodiment, the compounds described herein are suitable for the treatment, reducing the risk of, prevention, or alleviation of a symptom of pulmonary diseases or conditions caused by or associated with damages or injuries to the lungs. In one embodiment, damages or injuries to the lungs are a result of, for example, use of medications, substance abuse, or a medical condition. In one embodiment, damages or injuries to the lungs cause inflammation to the lungs.

In one embodiment, the compounds described herein are suitable for the treatment, reducing the risk of, prevention, or alleviation of a symptom of pulmonary diseases and conditions caused by or associated with narrowing of the pulmonary blood vessels. In one embodiment, narrowing of the pulmonary blood vessels is a result of, for example, use of medications, substance abuse, or a medical condition. In one embodiment, narrowing of the pulmonary blood vessels (e.g., arteries, veins, and capillaries) causes a decrease in the amount of blood that flows through the blood vessels. In one embodiment, narrowing of the pulmonary blood vessels causes an increase in the pressure of the blood that flows through pulmonary blood vessels.

Pulmonary diseases and conditions include, but are not limited to, chronic obstructive pulmonary disease (COPD), emphysema, asthma, idiopathic pulmonary fibrosis, pneumonia, tuberculosis, cystic fibrosis, bronchitis, pulmonary hypertension (e.g., Idiopathic Pulmonary Arterial Hypertension (IPAH) (also known as Primary Pulmonary Hypertension (PPH)) and Secondary Pulmonary Hypertension (SPH)), interstitial lung disease, and lung cancer.

An interstitial lung disease occurs when the interstitial tissue, which lines alveoli in the lungs, becomes scarred. Scarring causes inflammation of these tissues, affecting their ability to absorb oxygen. Causes of interstitial lung disease include, but are not limited to, environmental pollutants, lung tissue injury resulting from trauma or infection, and various connective tissue diseases.

Asthma affects millions of individuals around the world, from children to senior citizens. Asthma is caused by the contraction of the muscles in the airway, excessive mucus production, and swelling or inflammation of the airways or branches of the lungs. Airway constriction and inflammation results in reduced air flow to the lungs, which can often be noted by the wheezing sounds a person having an asthma attack may make. The treatment and management of asthma is determined on an individualized basis and is subject to considerations including the severity and frequency of asthma attacks experienced by the patient.

Bronchitis is a chronic infection of the bronchioles in the lungs. The bronchioles contain the alveoli, which are responsible for gas exchange during respiration. When bronchioles become infected, the immune system response results in swelling and increased mucous production in the airways, making it difficult to breathe. Bronchitis is also presented with a chronic, painful cough.

Emphysema also affects the alveoli, to the extent at which the cells that make them up are completely destroyed. Emphysema also destroys villi in the lungs. Villi are hair-like structures that push foreign substances out of the lungs. When they die, the lungs have an increased chance of infection. The effects of emphysema are permanent, and result in life long breathing difficulties.

One of the most common forms of COPD is emphysema. COPD damages the alveoli in the lungs, which are small air sacs found at the end of the lung branches that transport oxygen to the sacs. Weakened sac walls inhibit adequate oxygen flow into and out of the sacs, causing constant shortness of breath.

Cystic fibrosis is another common pulmonary disease that is hereditary in nature, meaning the condition is often passed down through family lines. A gene mutation causes the lungs to absorb excessive amounts of water and sodium, resulting in a buildup of fluids in the lungs that decreases their ability to absorb enough oxygen for optimal function. This condition gradually worsens as lung cells become increasingly damaged and eventually die.

Idiopathic pulmonary fibrosis (IPF) (or cryptogenic fibrosing alveolitis (CFA)) is a chronic, progressive form of lung disease characterized by fibrosis of the supporting framework (interstitium) of the lungs. By definition, the term is used only when the cause of the pulmonary fibrosis is unknown (“idiopathic”).

Tuberculosis is a disease that can spread from person to person through the air. It is a bacterial infection of the lungs. Anti-tuberculosis drugs are needed to kill bacteria very effectively. However, some strains of tuberculosis have developed a resistance to the anti-bacterial drugs used for treatment of the disease.

In one embodiment, the compounds described herein are suitable for the treatment, reducing the risk of, prevention, or alleviation of a symptom of an interstitial lung disease, asthma, bronchitis, COPD, emphysema, cystic fibrosis, IPF, tuberculosis, or pulmonary hypertension (e.g., IPAH, PPH, and SPH).

In one embodiment, the compounds described herein are suitable for the treatment, reducing the risk of, prevention, or alleviation of a symptom of COPD, emphysema, asthma, cystic fibrosis, or pulmonary hypertension (e.g., IPAH, PPH, and SPH).

In one embodiment, the compounds described herein are suitable for the treatment, reducing the risk of, prevention, or alleviation of pulmonary hypertension.

The compounds described herein are also suitable for reducing or suppressing inflammation in the lungs. “Suppressing”, “suppress”, or “suppression” means stopping the inflammation from occurring, worsening, persisting, lasting, or recurring. “Reducing”, “reduce”, or “reduction” means decreasing the severity, frequency, or length of the inflammation. Without the intention of being bound by the theory, compounds of invention reduce or suppress inflammation by decreasing the permeability of the lung and/or suppressing the movement of leukocytes or fibrocytes from circulation to inflamed tissues.

The compounds described herein are also suitable for promoting lung repair or recovery. “Promoting” or “promote” means reducing the time for the lung to repair or recover from injuries or damages to the lungs or increasing the extent of lung repair or recovery. In one embodiment, the compounds promote lung repair or recovery by reducing or suppressing inflammation in the lungs.

In one embodiment, the compounds described herein are FXR agonists. An FXR agonist means that the compounds of the invention mimic the action of the FXR receptor. For example, the compounds of the invention bind to the same receptor(s) or cellular target(s) as the FXR. For example, the compounds of the invention regulate or trigger the FXR signaling pathway. In one embodiment, the compounds described herein are suitable for the methods and uses of the invention through regulating or triggering the FXR signaling pathway.

In one embodiment, the compounds described herein decrease the number of fibrocytes moved to the lungs or to the location of injury in the lungs from circulation. In one embodiment, the compounds described herein decrease the amount of a protein, a peptide, or a chemokine produced by the fibrocytes in the lungs or at the location of injury in the lungs. In one embodiment, the compounds described herein decrease the amount of collagen I or CXCL12 produced by the fibrocytes in the lungs or at the location of injury in the lungs.

In one embodiment, the compounds described herein are suitable for the methods and uses of the invention by decreasing the number of fibrocytes moved to the lungs or to the location of injury in the lungs from circulation. In one embodiment, the compounds described herein are suitable for the methods and uses of the invention by decreasing the amount of a protein, a peptide, or a chemokine produced by the fibrocytes in the lungs or at the location of injury in the lungs. In one embodiment, the compounds described herein are suitable for the methods and uses of the invention by decreasing the amount of collagen I or CXCL12 produced by the fibrocytes in the lungs or at the location of injury in the lungs.

In one embodiment, the compounds described herein increase the expression of dimethylarginine dimethylaminohydrolase (DDAH). In one embodiment, the compounds described herein decrease the amount of ω-N°,N°-asymmetric dimethylarginine (ADMA).

In one embodiment, the compounds described herein are suitable for the methods and uses of the invention by increasing the expression of DDAH. In one embodiment, the compounds described herein are suitable for the methods and uses of the invention by decreasing the amount of ADMA.

In one embodiment, the compounds described herein decrease insulin sensitivity. In one embodiment, the compounds described herein are suitable for the methods and uses of the invention by decreasing insulin sensitivity.

In one embodiment, the compounds described herein regulate the expression of a gene involved in inflammation. In one embodiment, the compounds described herein decrease the expression of a pro-inflammatory factor. In one embodiment, the compounds described herein decrease the expression of IL-6 or monocyte chemoattractant protein-1 (MCP-1).

In one embodiment, the compounds described herein are suitable for the methods and uses of the invention by regulating the expression of a gene involved in inflammation. In one embodiment, the compounds described herein are suitable for the methods and uses of the invention by decreasing the expression of a pro-inflammatory factor. In one embodiment, the compounds described herein are suitable for the methods and uses of the invention by decreasing the expression of IL-6 or MCP-1.

In one embodiment, the compounds described herein regulate the expression of a gene involved in endothelium proliferation. In one embodiment, the compounds described herein increase the expression of an endothelium-proliferative factor. In one embodiment, the compounds described herein increase the expression of VEGF or ACE2.

In one embodiment, the compounds described herein are suitable for the methods and uses of the invention by regulating the expression of a gene involved in endothelium proliferation. In one embodiment, the compounds described herein are suitable for the methods and uses of the invention by increasing the expression of an endothelium-proliferative factor. In one embodiment, the compounds described herein are suitable for the methods and uses of the invention by increasing the expression of VEGF or ACE2.

In one embodiment, the compounds described herein regulate the expression of a gene involved in NO signaling. In one embodiment, the compounds described herein increase the expression of expression of a gene involved in NO signaling. In one embodiment, the compounds described herein increase the expression of GC1a3, GC1b3, PKG1, or PDE5.

In one embodiment, the compounds described herein are suitable for the methods and uses of the invention by regulating the expression of a gene involved in NO signaling. In one embodiment, the compounds described herein are suitable for the methods and uses of the invention by increasing the expression of a gene involved in NO signaling. In one embodiment, the compounds described herein are suitable for the methods and uses of the invention by increasing the expression of GC1a3, GC1b3, PKG1, or PDE5.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, n-propyl, isopropyl, n-butyl, iso-, sec- and tert-butyl, pentyl, and hexyl and the like.

Compounds within the scope of the instant invention may contain chiral centers and thus are capable of existing as racemates, racemic mixtures, diastereomers and single enantiomers. All such forms should be understood as within the scope of this invention.

The term “a compound of the invention” or “compounds of the invention” as used herein should be understood to include a compound of any of the formulae A, I, IA, II, and IIA or a pharmaceutically acceptable salt form and any compounds explicitly disclosed herein.

The compounds of the invention may be administered in the parent form or as a pharmaceutically acceptable salt thereof. Pharmaceutically acceptable salts can be prepared from a parent compound that contains basic or acidic moieties by conventional chemical methods. Acid addition salts would include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, diethanolamine, diethylamino, and triethylamino salts. For reviews on pharmaceutically acceptable salts, see S. M. Berge, L. D. Bighley and D. C. Monkhouse, Pharmaceutical Salts, J. Pharm. Sci., 66 (1977), 1-19 and P. H. Stahl and C. G. Wermuth (eds.), Pharmaceutical Salts: Properties, Selection, and Use, Weinheim, Germany: Wiley and Zurich: Verlag Helvetica Chimica Acta, 2002 [ISBN 3-906390-26-8], incorporated herein by reference. Any reference to the parent compound or a salt thereof should be understood to include all hydrates of the compound and all polymorphic forms of the parent compound.

The present invention provides methods of treating, reducing the risk of, preventing, or alleviating a symptom of a pulmonary disease or condition, or reducing or suppressing inflammation in the lung, or promoting lung repair in a subject. The compounds are useful in treating all forms of pulmonary diseases and conditions in which inflammation and/or activation of immune response is implicated. The compounds are also useful in treating all forms of pulmonary diseases and conditions in which an increase in NO production, downregulation of endothelin (ET)-1, decrease in the permeability of the lung, suppression of leukocyte or fibrocyte movement from circulation to inflamed tissues, or any combination thereof is implicated.

The present invention is also directed to a method for the manufacture of a medicament for the treatment, reducing the risk of, prevention, or alleviation of a pulmonary disease or condition, or for reducing or suppressing inflammation in the lung, or for promoting lung repair in subject.

As used herein, “subject” means a human or animal (in the case of an animal, more typically a mammal). In one aspect, the subject is a human. A subject can be considered to be in need of treatment.

As used herein, a “pharmaceutically-acceptable excipient” or a “pharmaceutically-acceptable carrier” means a pharmaceutically acceptable material, composition or vehicle involved in giving form or consistency to the pharmaceutical composition. Each excipient or carrier must be compatible with the other ingredients of the pharmaceutical composition when comingled such that interactions which would substantially reduce the efficacy of the compound of the invention when administered to a subject and interactions which would result in pharmaceutical compositions that are not pharmaceutically acceptable are avoided. In addition, each excipient or carrier must of course be of sufficiently high purity to render it pharmaceutically-acceptable.

A compound of the invention may be administered as a pharmaceutical composition. A compound of the invention and a pharmaceutically-acceptable excipient or excipients will typically be formulated into a dosage form adapted for administration to the subject by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, gels, and patches.

Suitable pharmaceutically acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the subject from one organ, or portion of the body, to another organ, or portion of the body. Certain pharmaceutically-acceptable excipients may be chosen for their ability to enhance compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

The compounds of the invention may also be coupled with soluble polymers as targetable drug carriers. Such polymers can include polyvinylpyrrolidone, pyran copolymer, polyhydroxypropylmethacrylamide-phenol, polyhydroxyethylaspartamide-phenol, or polyethyleneoxidepolylysine substituted with palmitoyl residues. Furthermore, the compounds of the invention may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

In one embodiment, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

In another embodiment, the invention is directed to a liquid oral dosage form. Oral liquids such as solution, syrups and elixirs can be prepared in dosage unit form so that a given quantity contains a predetermined amount of a compound of the invention. Syrups can be prepared by dissolving the compound of the invention in a suitably flavored aqueous solution, while elixirs are prepared through the use of a non-toxic alcoholic vehicle. Suspensions can be formulated by dispersing the compound of the invention in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another embodiment, the invention is directed to oral inhalation or intranasal administration. Appropriate dosage forms for such administration, such as an aerosol formulation or a metered dose inhaler, may be prepared by conventional techniques.

For administration by inhalation the compounds may be delivered in the form of an aerosol spray presentation from pressurized packs or a nebulizer, with the use of a suitable propellant, e.g. dichlorodifluoromethane, trichlorofluoromethane, dichlorotetrafluoroethane, a hydrofluoroalkane such as tetrafluoroethane or heptafluoropropane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of a compound of the invention and a suitable powder base such as lactose or starch.

Dry powder compositions for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di- or poly-saccharides (e.g., lactose or starch). Each capsule or cartridge may generally contain between 20 μg-10 mg of the compound of the invention, optionally in combination with another therapeutically active ingredient. Alternatively, the compound of the invention may be presented without excipients.

Suitably, the packing/medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant an inhaler having a reservoir form pack suitable for comprising multiple (un-metered doses) of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the subject for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In one embodiment, the carrier has a blister pack form, but it could also, for example, comprise a capsule-based pack form or a carrier onto which medicament has been applied by any suitable process including printing, painting and vacuum occlusion.

In the case of multi-dose delivery, the formulation can be pre-metered (e.g., as in Diskus, see GB 2242134, U.S. Pat. Nos. 6,632,666, 5,860,419, 5,873,360 and 5,590,645, or Diskhaler, see GB 2178965, 2129691 and 2169265, U.S. Pat. Nos. 4,778,054, 4,811,731, and 5,035,237, the disclosures of each of which are hereby incorporated by reference) or metered in use (e.g., as in Turbuhaler, see EP 69715 or in the devices described in U.S. Pat. No. 6,321,747, the disclosures of each of which are hereby incorporated by reference). An example of a unit-dose device is Rotahaler (see GB 2064336 and U.S. Pat. No. 4,353,656, the disclosures of each of which are hereby incorporated by reference).

The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of the invention, optionally combined with lactose. The strip is sufficiently flexible to be wound into a roll. The lid sheet and base sheet will preferably have leading end portions which are not sealed to one another and at least one of the said leading end portions is constructed to be attached to a winding means. Also, the hermetic seal between the base and lid sheets extends over their whole width. The lid sheet may preferably be peeled from the base sheet in a longitudinal direction from a first end of the said base sheet.

In one embodiment, the multi-dose pack is a blister pack comprising multiple blisters for containment of medicament in dry powder form. The blisters are typically arranged in regular fashion for ease of release of medicament therefrom.

In one embodiment, the multi-dose blister pack comprises plural blisters arranged in generally circular fashion on a disc-form blister pack. In another embodiment, the multi-dose blister pack is elongate in form, for example, comprising a strip or a tape.

In one embodiment, the multi-dose blister pack is defined between two members peelably secured to one another. U.S. Pat. Nos. 5,860,419, 5,873,360 and 5,590,645 describe medicament packs of this general type. The device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. Also, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the subject. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable. The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. In one aspect, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a “free flow aerosol valve” having a chamber and a valve stem extending into the chamber and movable relative to the chamber between dispensing and non-dispensing positions. The valve stem has a configuration and the chamber has an internal configuration such that a metered volume is defined there between and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085. Additionally, intra-nasal delivery of the present compounds is effective.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissue, the medicament must be capable of resisting those forces in the nasal passages that function to remove particles from the nose. Such forces, referred to as “mucociliary clearance”, are recognized as being extremely effective in removing particles from the nose in a rapid manner, for example, within 10-30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors.

A suitable dosing regime for the formulation of the present invention when administered to the nose would be for the subject to inhale deeply subsequent to the nasal cavity being cleared. During inhalation the formulation would be applied to one nostril while the other is manually compressed. This procedure would then be repeated for the other nostril.

A means for applying the formulation of the present invention to the nasal passages is by use of a pre-compression pump. For example, the pre-compression pump will be a VP7 model manufactured by Valois SA. Such a pump is beneficial as it will ensure that the formulation is not released until a sufficient force has been applied, otherwise smaller doses may be applied. Another advantage of the pre-compression pump is that atomization of the spray is ensured as it will not release the formulation until the threshold pressure for effectively atomizing the spray has been achieved. Typically, the VP7 model may be used with a bottle capable of holding 10-50 ml of a formulation. Each spray will typically deliver 50-100 μl of such a formulation, therefore, the VP7 model is capable of providing at least 100 metered doses.

Spray compositions for topical delivery to the lung by inhalation may for example be formulated as aqueous solutions or suspensions or as aerosols delivered from pressurized packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetrafluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants, e.g., oleic acid or lecithin and cosolvents, e.g. ethanol. Pressurized formulations will generally be retained in a canister (e.g., an aluminum canister) closed with a valve (e.g., a metering valve) and fitted into an actuator provided with a mouthpiece.

Medicaments for administration by inhalation desirably have a controlled particle size. The optimum particle size for inhalation into the bronchial system is usually 1-10 mm, preferably 2-5 mm. Particles having a size above 20 mm are generally too large when inhaled to reach the small airways. To achieve these particle sizes the particles of the active ingredient as produced may be size reduced by conventional means e.g., by micronization. The desired fraction may be separated out by air classification or sieving. Suitably, the particles will be crystalline in form. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 mm and not less than 15% will have a MMD of less than 15 mm.

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulization may be formulated with an aqueous vehicle with the addition of agents such as acid or alkali, buffer salts, isotonicity adjusting agents or antimicrobials. They may be sterilized by filtration or heating in an autoclave, or presented as a non-sterile product.

Pharmaceutical compositions adapted for transdermal administration may be presented as discrete patches intended to remain in intimate contact with the epidermis of the subject for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

For treatments of external tissues, for example mouth and skin, the compositions may be applied as a topical ointment or cream. When formulated in an ointment, the compound of the invention may be employed with either a paraffinic or a water-miscible ointment base. Alternatively, the compound of the invention may be formulated in a cream with an oil-in-water cream base or a water-in-oil base.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compounds of the invention can be administered to a subject before or after a lung injury. In one embodiment, the compounds of the invention are administered to a subject after a lung injury, for example, a lung injury induced or caused by inflammation, autoimmune diseases such as scleroderma and rheumatoid arthritis, Acute Lung Injury (ALI), Acute Respiratory Distress Syndrome (ARDS), birth defects of the heart, blood clots in the lungs (pulmonary embolism), congestive heart failure, heart valve disease, HIV infection, extended periods of low oxygen levels in the blood, various medications and substances of abuse, and/or obstructive sleep apnea. In one embodiment, the compounds of the invention are administered to a subject after a lung injury is induced or caused by inflammation, autoimmune diseases such as scleroderma and rheumatoid arthritis, ALI, and/or ARDS. In one embodiment, the compounds of the invention are administered to a subject after a lung injury is induced or caused by medications and other substances which are capable of causing lung injuries.

Methods and uses of the invention can be carried out by using techniques and materials known in the art. For example, methods and uses of the invention can be carried out with techniques and material described in Ghebremariam Y. T. et al., PLoS One 8: e60653 (2013), Cowan K. N. et al., Nat Med 6:698-702 (2000), and Sakuma F. et al., Lung 177:77-88 (1999). Additional techniques and materials for assessing or evaluating the compounds of the invention are known in the art. For example, the compounds of the invention can be assessed or evaluated by measuring a subject's response to the compounds. In one example, the compounds of the invention can be assessed or evaluated by measuring in a subject the amount or concentration of a substance (e.g., a protein, a peptide, a chemokine, a DNA, a RNA, an mRNA, a gene, a metabolite, and a cell) whose amount or concentration in the subject is affected (e.g., increased, upregulated, elevated, decreased, downregulated, and reduced) by the compounds of the invention, for example, through the FXR signaling pathway. In one example, the compounds of the invention can be assessed or evaluated by measuring the amount of leukocytes and/or fibrocytes moving to the lungs of the subject. In another example, the compounds of the invention can be assessed or evaluated by measuring the amount or concentration of a protein, a peptide, or a chemokine (e.g., collagen, CXCL12, dimethylarginine dimethylaminohydrolase (DDAH), and ω-N°,N°-asymmetric dimethylarginine (ADMA)) in the subject (e.g., in the lungs of the subject). In another example, the compounds of the invention can be assessed or evaluated by measuring the amount or concentration of a gene involved in inflammation, endothelium proliferation, or NO signaling (e.g., a pro-inflammatory factor (e.g., IL-6 and MCP-1), an endothelium-proliferative factor (e.g., VEGF and ACE2), GC1a3, GC1b3, PKG1, or PDE5).

### Equivalents

Those skilled in the art will recognize, or be able to ascertain using no more than routine experimentation, many equivalents to the specific embodiments and methods described herein. Such equivalents are intended to be encompassed by the scope of the present invention.

All patents, patent applications, and literature references cited herein are hereby expressly incorporated by reference.

The following Examples are illustrative and should not be interpreted in any way so as to limit the scope of the invention.

### EXAMPLES

### Example 1

### Preparation of Compound 1

a) Preparation of Methyl 3α-hydroxy-7-keto-5β-cholanate (III). 17.0 kg of 3α-hydroxy-7-keto-5β-cholanic acid, 68 kg of methanol and 0.17 kg of methansulphonic acid were charged into a reactor. The reaction mixture was then heated to 30-60° C. for 1 hour and 25.5 kg of demineralised water was added. The mixture obtained was then stirred, cooled to 20-25° C. until a good precipitation was obtained, then cooled further to 0-15° C. The precipitate was filtered and washed with a mixture of water and methanol and further dried in an oven at about 40° C. 15 kg of methyl 3α-hydroxy-7-keto-5β-cholanate (III) was thus obtained. The stoichiometric yield was 85.2%.

b) Preparation of Methyl 3α-trimethylsiloxy-7-keto-5β-cholanate (IV). 15.0 kg of methyl 3α-hydroxy-7-keto-5β-cholanate, 45 kg of toluene, 7.5 kg of triethylamine, and 7.5 kg of trimethylchlorosilane were charged into a reactor. The mixture was heated to 70-80° C. and was kept under stirring at that temperature for about 1 hour, then 37.5 kg of water was added and the mixture was stirred at 15-20° C. The lower aqueous phase was then separated and eliminated. The organic phase was concentrated until an oily residue was obtained, to which 15 kg of tetrahydrofuran was added. The solution thus obtained containing methyl 3α-trimethylsiloxy-7-keto-5β-cholanate (IV) was used in the following stage (c).

c) Preparation of methyl 3α,7α-di-trimethylsililoxy-5β-cholanate (V). 30 kg of tetrahydrofuran was loaded in a reaction vessel, then the mixture was brought to a temperature between −90° and −60° C. 9.8 kg of 100% lithium diisopropylamide and 9.3 kg of trimethylchlorosilane were added, and the whole solution of tetrahydrofuran prepared in (b) and containing methyl 3α-trimethylsiloxy-7-keto-5β-cholanate was poured. The mixture was then stirred for about 1 hour at a temperature between −60 and −90° C. for 1 hour. A solution of 4.50 kg of sodium bicarbonate and 60 kg of water was then poured and the mixture was stirred at 0-10° C., and the lower aqueous phase was separated and eliminated. The lower phase was then concentrated until an oily residue was obtained, to which 45.0 kg of methylene chloride was added. The solution of methyl 3α,7α-di-trimethylsililoxy-5β-cholanate thus obtained was sent to the next stage (d).

d) Preparation of methyl 3α-hydroxy-6-ethylidene-7-keto-5β-cholanate (VI). The whole solution of methyl 3α,7α-di-trimethylsililoxy-5β-cholanate in methylene chloride coming from the preceding step was charged into a reactor and cooled to between −90 and −60° C. 1.97 kg of acetaldehyde and 5.5 kg of boron trifluoride etherate were then added. The reaction mixture was kept under stirring at the above temperature for 2-4 hours, after which it was heated to 30-35° C. and kept at that temperature for about 2-4 hours. Then 60 kg of water was added. The mixture obtained was stirred and the aqueous phase was separated. The solution thus obtained containing methyl 3α-hydroxy-6-ethylidene-7-keto-5β-cholanate was used in the next step.

e) Preparation of 3α-hydroxy-6-ethylidene-7-keto-5β-cholanic (VII) acid. The solution of methyl 3a-hydroxy-6-ethylidene-7-keto-5β-cholanate in methylene chloride obtained in the previous step was charged into a reactor. The solvent was then removed by distillation until an oily residue was obtained, to which 15 kg of methanol was added. The reaction mixture was then heated to 45-50° C. and 7.5 kg of 30% sodium hydroxide was added, and the reaction mixture was kept at the above temperature for about 1 hour. Then 30 kg of water was added, followed by 45.0 kg of methylene chloride and 7.5 kg of 85% phosphoric acid. The lower organic phase was separated and the aqueous phase was eliminated subsequently. The solvent was removed from the organic phase by distillation until a pasty residue was obtained. About 37.5 kg of ethyl acetate was added to the residue and the mixture was heated to 65-75° C., then cooled to 10-35° C. The precipitate was obtained, filtered and washed with ethyl acetate, and was dried. 8.0 kg of 3α-hydroxy-6-ethyliden-7-keto-5β-cholanic acid was obtained, with a stoichiometric yield of 51.8% calculated on methyl 3α-hydroxy-7-keto-5(3-cholanate.

f) Preparation of 3α-hydroxy-6β-ethyl-7-keto-5β-cholanic acid (IX). 8.0 kg of 3α-hydroxy-6-ethylidene-7-keto-5β-cholanic acid, 48.0 kg of water, 5.1 kg of 30% sodium hydroxide, 0.80 kg of 5% palladium/carbon were charged into a reactor. The reaction mixture was hydrogenated at a pressure between 1 and 3 atmospheres, until the hydrogen absorption was no longer noted.

g) Preparation of 3α-hydroxy-6α-ethyl-7-keto-5β-cholanic acid (IX). At the end of the reaction the mixture was heated to 95-105° C. and is kept at that temperature for a few hours to allow the 3α-hydroxy-6β-ethyl-7-keto-5β-cholanic acid (VIII) to convert into the corresponding epimer of the desired 3α-hydroxy-6α-ethyl-7-keto-5β-cholanic acid (IX). The suspension was filtered, and the catalyst was recovered. 5.1 kg of 85% phosphoric acid 9.6 kg of ethyl acetate were added to the filtered solution and the reaction mixture was heated to a temperature between 40 and 70° C. It was cooled to a temperature between 0 and 30° C. and the precipitate was recovered by filtration. After washing with ethyl acetate, the precipitate was dried in an oven at 65° C. 5.0 kg of 3α-hydroxy-6α-ethyl-7-keto-5β-cholanic acid was obtained. Stoichiometric yield: 62.2%. m.p. 185-188° C.

h) Preparation of 3α,7α-dihydroxy-6α-ethyl-5β-cholanic acid. 5.0 kg of 3α-hydroxy-6α-ethyl-7-keto-5β-cholanic acid, 5.0 kg of water, 2.50 kg of sodium hydroxide were loaded in a reactor. The mixture was then heated to 70-105° C. and a mixture of sodium borohydride dissolved in 2.50 kg of water was poured, the mixture was then kept warm for 1 hour, cooled to room temperature, and 10.0 kg of demineralised water, 15.0 kg of methylene chloride and 3.00 kg of 85% phosphoric acid were added. The mixture was stirred, the lower organic phase was separated and the aqueous phase was removed. Crystallization of the crude product was obtained by cooling the organic solution. This product was dissolved in 50 kg of demineralised water and 1.10 kg of 30% ammonia. The mixture was then stirred until a complete solution was obtained. The mixture was kept at 20-50° C., and 1.50 kg of phosphoric acid was poured. The precipitated mixture was stirred at a temperature between 20 and 50° C., then the precipitate was recovered by filtration, washed with water and dried. 4.50 kg of 3α,7α-di-hydroxy-6α-ethyl-5β-cholanic acid. Stoichiometric yield: 89.6%.

### Example 2

### Preparation of Compounds 2-4

3α-Tetrahydropyranyloxy-7-keto-5β-cholan-24-oic Acid (2A). 3,4-dihydro-2H-pyrane (1.74 ml, 19 mmol) in dioxane (12 ml) was dropped slowly to a solution of p-toluenesulfonic acid (115 mg, 0.6 ml) and 6α-ethyl-7-ketolithocholic acid (5.0 g, 12 mmol) in dioxane (55 ml). The reaction mixture was stirred at room temperature for 2 hours. Water (40 ml) was then added, and the mixture was partially concentrated under vacuum and extracted with EtOAc (4 times/25 ml). The combined organic fractions were washed with brine (1 times/50 ml), dried over anhydrous Na₂SO₄ and evaporated under vacuum to afford 6 g of compound 2A. The crude derivative was used for the next step without further purification.

3α-Tetrahydropyranyloxy-6α-ethyl-7-keto-24-nor-5β-cholan-23-iodide (3A). Under irradiation with a 300 w tungsten lamp, iodine (5 g, 20 mmol) in CCl₄ (75 ml) was added dropwise to a solution of 2 (5.5 g, 11 mmol) and lead tetra-acetate (4.9 g, 11 mmol) in CCl₄ (200 ml). The reaction mixture was stirred until the color was permanent (18 h). The mixture was cooled and filtered on Celite®. The organic phase was washed with a 5% Na₂S₂O₃ solution, 5% NaOH, brine (15 ml), dried over anhydrous Na₂SO₄ and evaporated under vacuum. The residue was purified by silica gel flash chromatography using a mixture of light petroleum/EtOAc 95/5 as mobile phase to give 4.6 g of compound 3A (40% yield).

3α-hydroxy-6α-ethyl-7-keto-24-nor-5β-cholan-23-iodide (4A). The compound 3A (2.2 g, 3.8 mmol) was stirred in a solution of HCl 37% in THF (50 ml) overnight at room temperature. The reaction mixture was washed with a saturated solution of NaHCO₃ (20 ml), H₂O (20 ml), and brine (20 ml), dried over Na₂SO₄, and evaporated under vacuum to afford 1.4 g of compound 4A (80% yield). The crude derivative was used for the next step without further purification.

3α-tert-Buthyldimethylsilyloxy-6α-ethyl-7-keto-24-nor-5β-cholan-23-iodide (5A). To a solution of 4A (1.4 g, 2.8 mmol) in CH₂Cl₂ (30 ml), tert-butyldimethylsilylchloride (496 mg, 3.22 mmol) and imidazole (230 mg, 3.36 mmol) were added and the mixture was stirred overnight at room temperature. The reaction mixture was washed with a saturated solution of NaHCO3 (30 ml), brine (30 ml), and dried over anhydrous Na₂SO₄. The organic phase was evaporated under vacuum to afford 1.5 g of compound 5A (87% yield). The crude derivative was used for the next step without further purification.

3α-tert-Buthyldimethylsilyloxy-6α-ethyl-7-keto-24-nor-5β-cholan-23-ole (6A). To a solution of 5 (1.2 g, 1.96 mmol) in acetone (12 ml), Ag₂CO₃ (1.1 g, 3.9 mmol) was added. The reaction mixture was refluxed overnight and then cooled to r.t., filtered on Celite® washed with acetone and the combined organic phases were concentrated to yield 1 g of compound 6A. The crude derivative was used for the next step without further purification.

3α-tert-Buthyldimethylsilyloxy-7α-hydroxy-6α-ethyl-24-nor-5β-cholan-23-ole (7A). To a solution of 6A (1 g, 1.96 mmol) in a mixture of THF (50 ml) and H₂O (12.5 ml), NaBH₄ (740 mg, 19.6 mmol) was added and the mixture was stirred at room temperature for 1 hours and 30 minutes. The reaction solution was partially concentrated under vacuum and extracted with CHCl₃ (3 times/20 ml). The combined organic layers were washed with brine (1 time /50 ml), dried over anhydrous Na₂SO₄, and evaporated under vacuum. The crude residue was purified by silica gel flash chromatography using a mixture of CH₂Cl₂:MeOH 99:1 as mobile phase to give 0.8 g of 7A (81% yield).

3α-tert-Butyldimethylsilyloxy-7α-hydroxy-6α-ethyl-24-nor-5β-cholan-23-sulphate triethyl ammonium salt (8A). To a solution of 7A (0.5 g, 0.99 mmol) in THF (7 ml) cooled at −3° C., Et₃N (0.3 ml, 2.1 mmol) was added and the resulting mixture was stirred for 10 min. ClSO₃H (0.1 ml, 1.5 mmol) was added and the mixture was stirred overnight at room temperature. Water (10 ml) was then added and the mixture was extracted with CH₂Cl₂ (3 times/15 ml), dried over anhydride Na₂SO₄, and evaporated under vacuum. The crude sulphate derivative was used for the next step without further purification.

3α,7α,23-trihydroxy-6α-ethyl-24-nor-5β-cholan-23-sulphate triethyl ammonium salt (Compound 4). To a solution of 8A (0.5 g, 0.77 mmol) in acetone (8 ml), PdCl₂(CH₃CN)₂ (10 mg, 0.05 eq) was added and the mixture was stirred at room temperature for 3 hours. The reaction mixture was filtered, concentrated under vacuum and purified by medium pressure Lichroprep RP-8 using a MeOH/H₂O 8/2 mixture as mobile phase to afford 0.115 g of 4, mp 118-121° C.

3α,7α,23-trihydroxy-6α-ethyl-24-nor-5β-cholan-23-sulphate sodium salt (Compound 3). To a solution of 8A (0.4 g, 0.72 mmol) in a mixture of acetone (4 ml) and H₂O (0.08 ml), PdCl₂(CH₃CN)₂ (10 mg, 0.05 eq) was added and the resulting mixture was stirred at room temperature for 3 hours. The reaction mixture was filtered over Celite® and concentrated under vacuum. The resulting residue was treated with a methanolic solution of 10% NaOH for 2 h. The resulting mixture was concentrated under vacuum and submitted to liquid medium pressure purification using a mixture of CH₃OH/H₂O (7:3) as mobile phase to afford 0.09 g of 3 (25% yield).

### Example 3

### Method of FXR Activation with Compound 1 Ameliorates the Pulmonary Fibrosis in the Murine Bleomycin-Induced Model

The murine model of bleomycin-induced pulmunary fibrosis was induced in C57Bl/6 wild-type and FXR^{-/-} mice (females, 6-8 weeks old). Groups of treatment included:
WT mice: A—saline (day 0); B—bleomycin (day 0); C—bleomycin (day 0)-Compound 1 (also referred to as 6ECDCA) (5 mg/kg, daily)
FXR^{-/-}]mice: D—saline (day 0); E—bleomycin (day 0); F—bleomycin (day 0) Compound 1 (also referred to as 6ECDCA) (5 mg/kg, daily).

After 22 days, the mice were sacrificed and the subsequent analyses were performed: (1) H&E and sinus red staining on lung sections; (2) Quantification of collagen I into the lung by qRT-PCR and Sircol collagen assay; (3) FXR, SHP and CXCL12 mRNA quantification by qRT-PCR; and (4) CXCL12 protein quantification by ELISA on lung homogenates.

Without wishing to be bound by theory, it is thought that the compounds of the invention activate FXR and effect amelioration of pulmonary fibroris by (1) decreasing the production of collagen I by resident fibroblasts and (2) decreasing the production of CXCL12 by resident fibroblasts and subsequently decreasing recruitment of circulating fibrocytes to the site of injury.

FXR activation reduced the production of collagen I and CXCR12 by resident fibroblasts. Specifically, after a 24 hour period of starving, cells from the murine lung fibroblast cell line (ATCC number CCL-206) were stimulated with or without TGFβ1 (10 ng/ml) and 6ECDCA (10 μM) for 24 hours and then the FXR, SHP and Col I gene expression was analyzed by qRT-PCR. After a 24 hours period of starving, cells from the murine lung fibroblast cell line (ATCC number CCL-206) were stimulated with or without TNFα (10 ng/ml) and 6ECDCA (10 μM) for 24 hours and then the FXR, SHP and CXCL12 gene expression was analyzed by qRT-PCR.

The downregulation of Col I and CXCL12 induced by 6ECDCA was FXR-mediated. After an overnight period of starving, murine lung fibroblast cell line (ATCC number CCL-206) was stimulated with or without TGFβ1 (10 ng/ml) and 6ECDCA (1 μM) for 24 hours—with and without block of the FXR by siRNA—and then the FXR, SHP, Col I and CXCL12 gene expression was analyzed by qRT-PCR. CXCL12 and Col I secretion in the supernatants was analyzed by ELISA and the Sircol collagen assay, respectively.

The downregulation of Col I and CXCL12 induced by 6ECDCA was SHP-mediated. Specifically, transfection of pulmonary fibroblasts with vector carrying the HA-SHP chimera was carried out to induce a SHP overexpression (WB analysis of the HA-SHP expression). Col I and CXCL12 expression (by qRT-PCR) was analyzed at baseline and after stimulation with TGFβ1. SHP overexpression was sufficient to down-regulate the Col I and CXCL12 expression, basically and after stimulation with TGFβ1.

After an overnight period of starving, murine lung fibroblast cell line (ATCC number CCL-206) were stimulated with/without TGFβ1 (10 ng/ml) and 6ECDCA (1 μM) for 24 hours—with and without block of the SHP by siRNA—and then the FXR, SHP, Col I and CXCL12 gene expression was analyzed by qRT-PCR. CXCL12 and Col I secretion in the supernatants was analyzed by ELISA and the Sircol collagen assay, respectively.

A decrease in the CXCL12-mediated recruitment of circulating fibrocytes in the site of injury was measured. The amount of murine CD45+/Col I+/CXCR+ fibrocytes into the lungs of mice with bleomycin-induced pulmonary fibrosis (treated and untreated) was determined by FACS analysis.

The isolation of human circulating fibrocytes was carried out as follows: PBMCs were isolated from leukopheresis packs, then cultured in DMEM with 20% FCS for 1 week. The fibrocytes were purified by immunomagnetic negative selection to deplete B and T lymphocytes and monocytes/macrophages (Dynabead method). Purified fibrocytes were returned to culture for an additional 5 days before FACS analysis of purity (CD45+/Col I+/CXCR4+]cells) and their infusion in SCID mice.

Specifically, the induction of bleomycin pulmonary fibrosis, 6ECDCA treatment and analysis of human fibrocyte infiltration into the lungs was carried out as follows:

Induction of pulmonary fibrosis by intratracheal injection of bleomycin in SCID mice: After 4 days, all mice received tail-vein injections of 1*10⁶ purified human fibrocytes. Groups: A saline solution; B bleomycin; C bleomycin+6ECDCA; D bleomycin+anti-murine CXCL12 antibody. After a further 4 days, the amount of human CD45+/Col I+/CXCR4+ fibrocytes into the lungs was analyzed by FACS analysis.

Further analysis: H&E and sinus red staining on lung sections; quantification of collagen I into the lung by qRT-PCR and Sircol collagen assay; FXR,

SHP and CXCL12 mRNA quantification by qRT-PCR; CXCL12 protein quantification by ELISA on lung homogenates

### Example 4

### Studies of 6ECDCA on Dahl Salt-Sensitive Rats

ADMA (ω-N°,N°-asymmetric dimethylarginine) is a major cause of endothelial dysfunction, which leads to plaque formation, progression, and rupture. See Coke, Circulation, 109 (2004): 1813-1819. Many diseases are associated with elevated ADMA levels. These diseases include e.g., retinal venous occlusive disease, early autosomal dominant polycystic kidney disease, proteinuria, secondary amyloidosis and endothelial dysfunction, children with sporadic focal segmental glomerulosclerosis, pre-eclampsia, chronic thromboembolic pulmonary hypertension, uncomplicated type 1 diabetes, pulmonary hypertension, sickle cell disease, depression, congestive heart failure, alzheimer's disease (reduced ADMA levels also reported), renal disease associated with cardiovascular disease, hypercholesterolaemia, hyperhomocysteinaemia, hypertension, atherosclerosis, and stroke. DDAH (dimethylarginine dimethylaminohydrolase), by metabolizing ADMA, has beneficial effects on blood pressure and insulin resistance. DDAH overexpression can increase NO synthesis and reduces blood pressure. H. Dayoub et al., Circulation 108 (24): 3042-3047. DDAH overexpression can also enhance insulin sensitivity. Sydow et al., Arterioscler Throm Vasc Biol. 28 (2008) : 692-697. ADMA plays a role in salt-sensitive hypertension. H. Matsuoka et al., Hypertension 1997, 29: 242-247.

The experiment below demonstrated that 6ECDCA can enhance insulin sensitivity and reduce blood pressure in the salt-sensitive hypertensive rat, by increasing DDAH expression and reducing ADMA levels.

The rodent model for salt-sensitive hypertension was a DSS (Dahl salt-sensitive) rat (e.g., Rapp). The DSS rat (8% NaCl diet) exhibits certain characteristics including e.g., albuminuria, aortic and cardiac hypertrophy, heart failure with pulmonary congestion, insulin resistance, hyperinsulinemia, hypertriglyceridemia, and hyperlipidemia. Specifically, the rodent models used for the study were male 4-week old DSS rats (e.g., SS/JrHsd) from Harlan Laboratories. The normal diet of the DSS rats was 0.49% NaCl and the high salt diet was 8% NaCl (e.g., Teklad Custom Research Diet). The rats were divided into four groups (herein referred to as DSS study rats):
Group 1; Normal salt diet (1% Methylcellulose) (N=6)
Group 2; High-salt diet (Vehicle; 1% MC) (N=9)
Group 3; High-salt diet (6ECDCA 10mg/kg/day) (N=6)
Group 4; High-salt diet (6ECDCA 30mg/kg/day) (N=9)

The following analyses were carried out: ADMA and NO levels in serum and urine and tissues (e.g., liver, muscle and kidney), blood pressure and heart rate, fasting blood glucose and insulin (HOMA-IR), ipGTT (IR-index), and urinary protein and creatinine. Additional studies such as electrolytes (Na+), histological analysis and TGFbeta expression in kidney, DDAH expression and activity in liver, skeletal muscle and kidney, and Akt-phosphorylation level in liver, skeletal muscle and kidney can also be carried out.

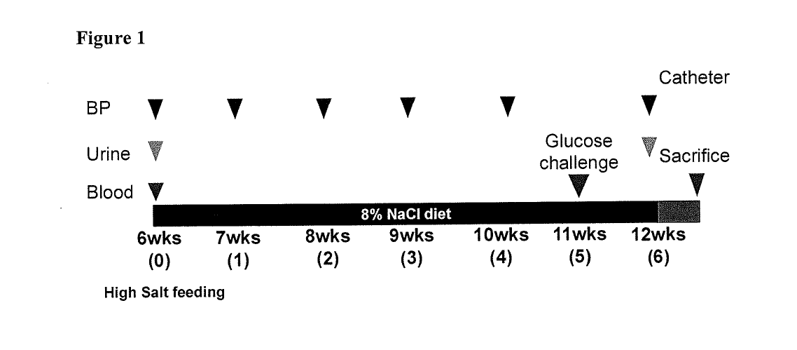

ECDCA was administered orally daily for 6-weeks. Blood and urine collections were performed at week 0 and week 6 and analyzed by methods known in the art. Blood pressure measurement was performed at week 0, 1, 2, 4 and 6 by tail cuff devices, which was performed on conscious models and was non-invasive. The blood pressure was also obtained via catheter, which was performed when the model was sacrificed. The glucose challenge test was performed at week 5. Renal function was analyzed by measuring the urinary volume, protein and creatinine in a 24 h-urine sample. Histological analysis was performed using the Masson&Trichrome staining. Insulin resistance was analyzed using the ipGTT test and the HOMA/IR index. The ADMA and NO levels were detected in blood concentration and urinary excretion (FIG. 1).

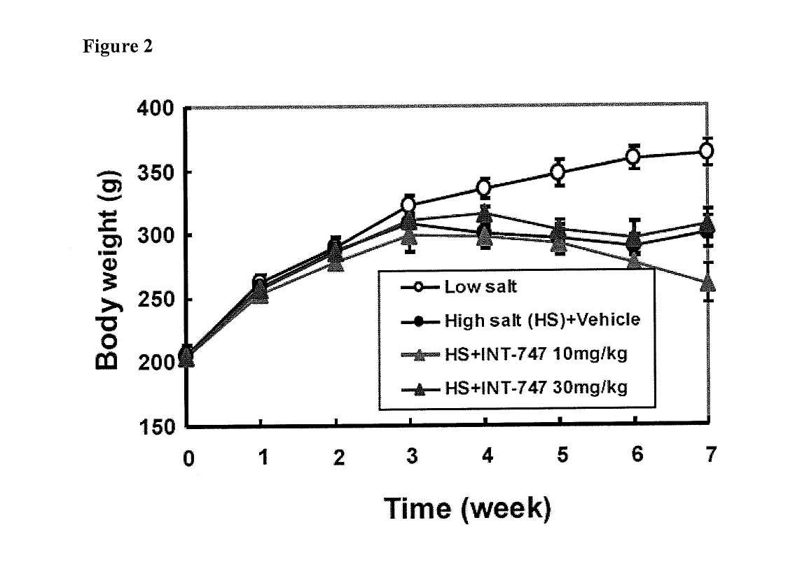

In a seven week study of (1) a low salt model, (2) a high salt (HS)+vehicle, (3) HS+6ECDCA at 10 mg/kg, and (4) HS+6ECDCA at 30 mg/kg, 6ECDCA did not affect the body weight of rats with high salt diet (FIG. 2).

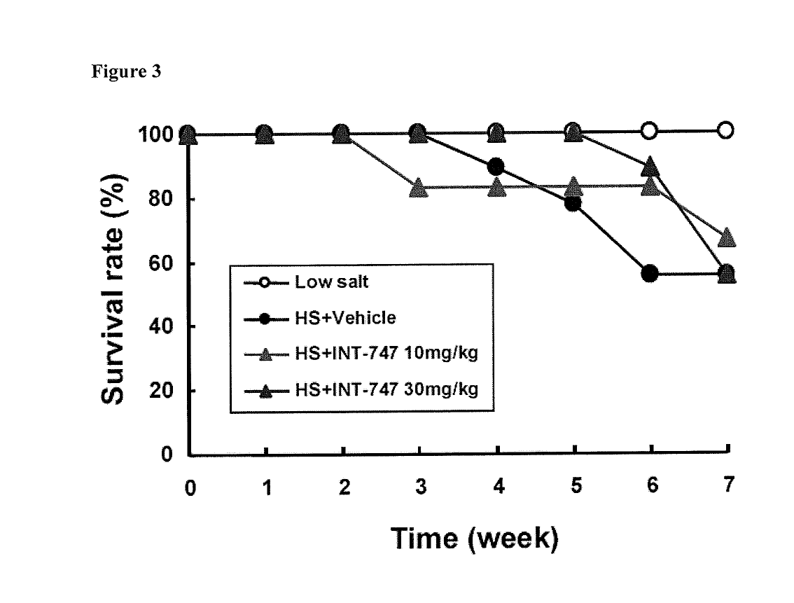

It has been previously reported that high salt diet in the RAPP model causes mortality. FIG. 3 is a graph indicating the survival rate (%) of DSS study rats versus time (week).

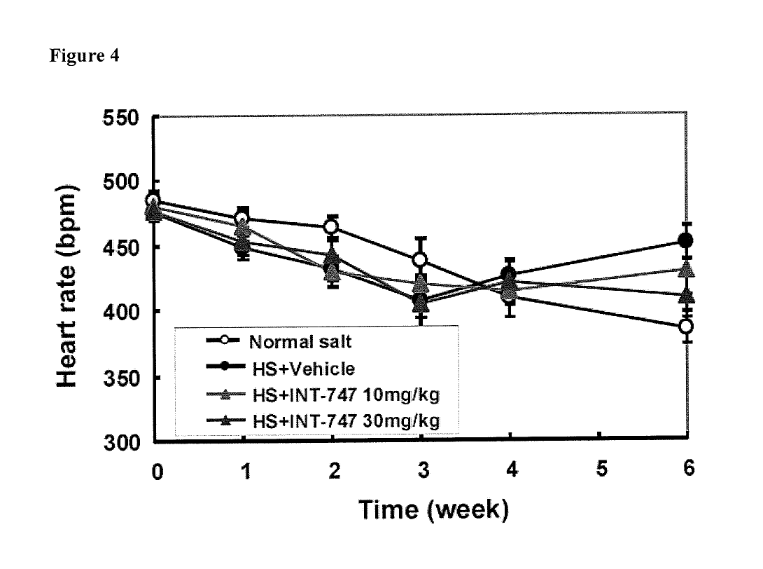

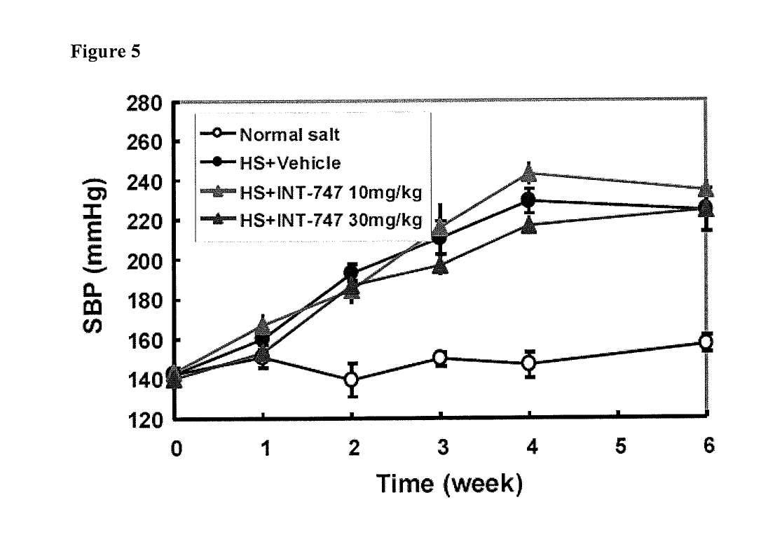

It was shown that high salt diet increased blood pressure and 6ECDCA treatment did not affect heart rate blood pressure or reduce blood pressure (FIGS. 4 and 5).

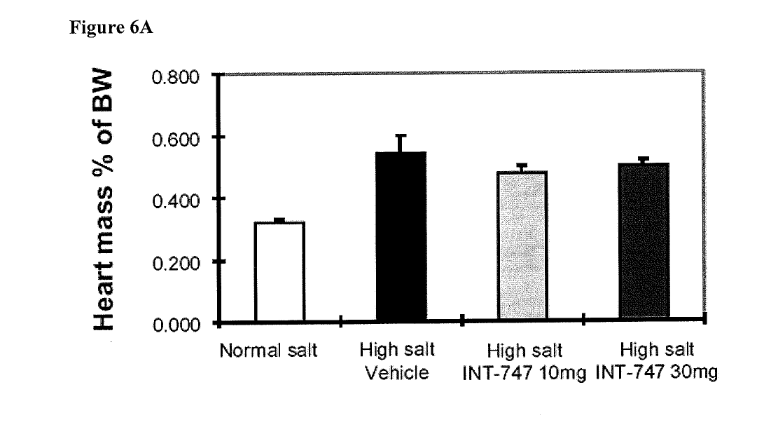

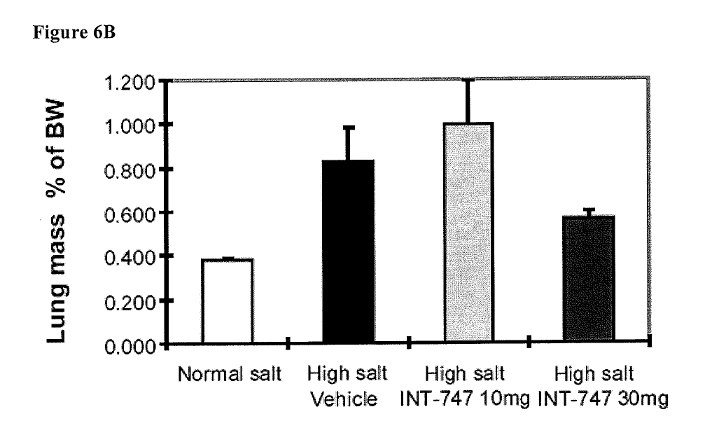

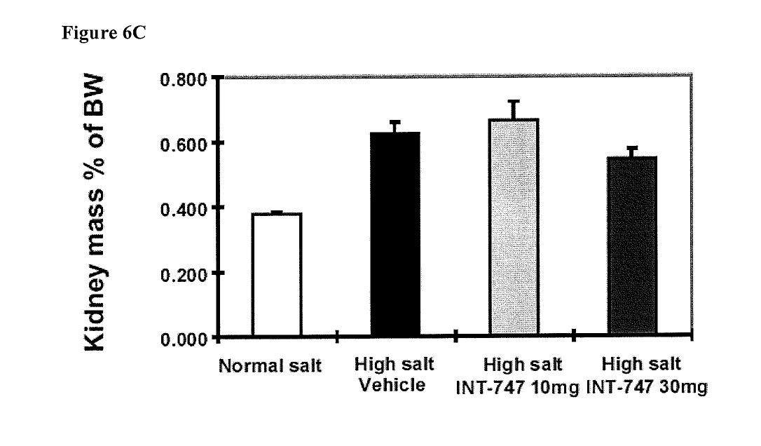

High salt feeding is known to induce cardiac hypertrophy, pulmonary congestion and renal fibrosis. 6ECDCA reduced lung weight at the dose of 30 mg/kg, suggesting that 6ECDCA is protective against pulmonary congestion (FIGS. 6A-6C).

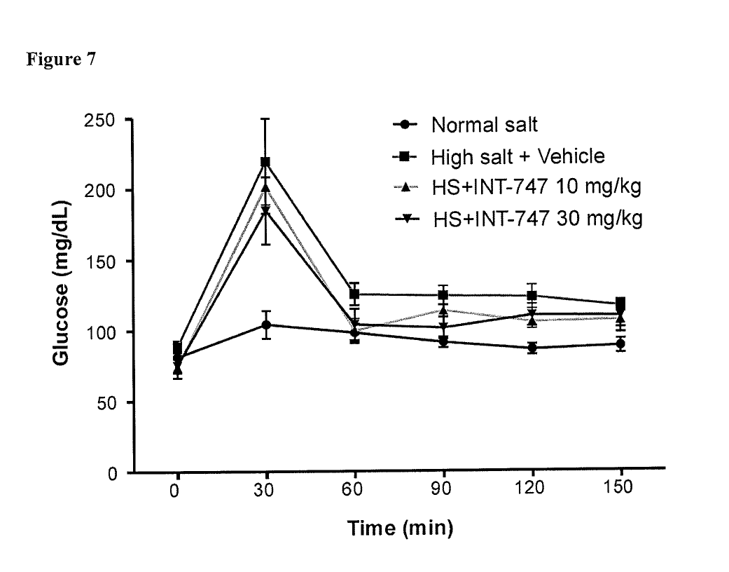

The fasting blood glucose concentration over time during glucose tolerance test (GTT) in DSS rats was measured (FIG. 7). FIG. 7 is a graph indicating the fasting blood glucose concentration (mg/dL) over time (min) during GTT in DSS study rats. Values are mean±SEM for: Control (n=6); Vehicle (n=7); 6ECDCA at 10 mg/kg (n=5) and 6ECDCA at 30 mg/kg (n=9).

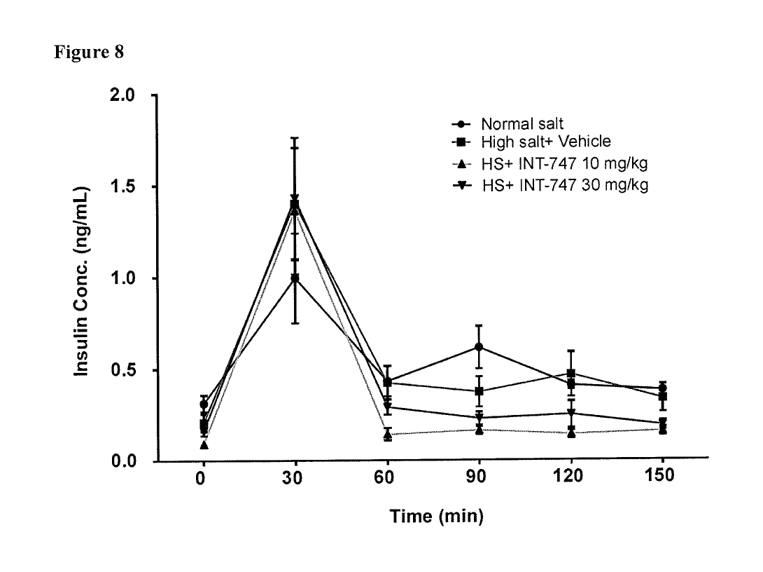

The fasting plasma insulin concentration over time during GTT in DSS study rats was measured (FIG. 8). FIG. 8 is a graph indicating the fasting plasma insulin concentration (ng/mL) over time (min) during GTT in DSS study rats. Values are mean±SEM for: Control (n=6); Vehicle (n=7); 6ECDCA at 10 mg/kg (n=5) and 6ECDCA at 30 mg/kg (n=9).

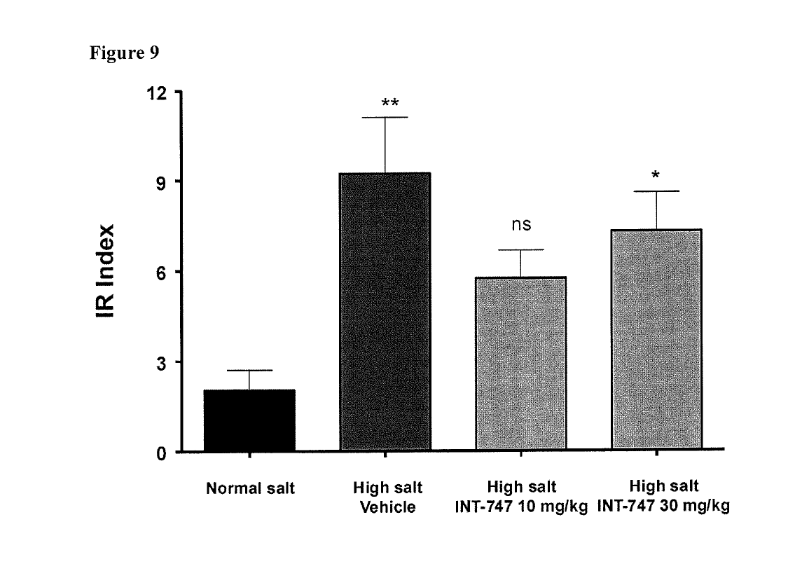

In the assessment of insulin sensitivity (Insulin Resistance-Index), 6ECDCA reversed insulin resistance induced by a high salt diet. The IR-index is the product of the average elevation in plasma glucose concentration over the fasting value times the average plasma insulin concentration (FIG. 9). FIG. 9 is a histogram indicating the insulin sensitivity using insulin resistance (IR) index in DSS study rats. Values are mean±SEM for: Control (n=6); Vehicle (n=6); 6ECDCA at 10 mg/kg (n=5) and 6ECDCA at 30 mg/kg (n=9).

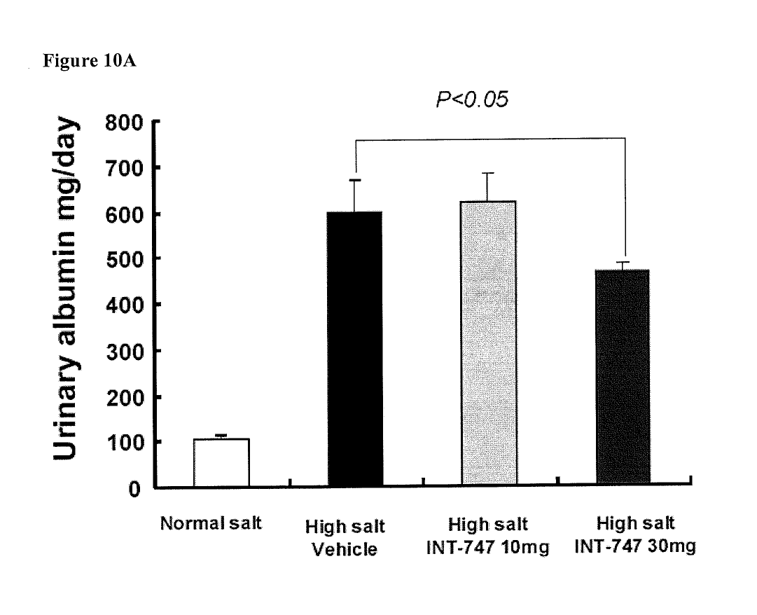

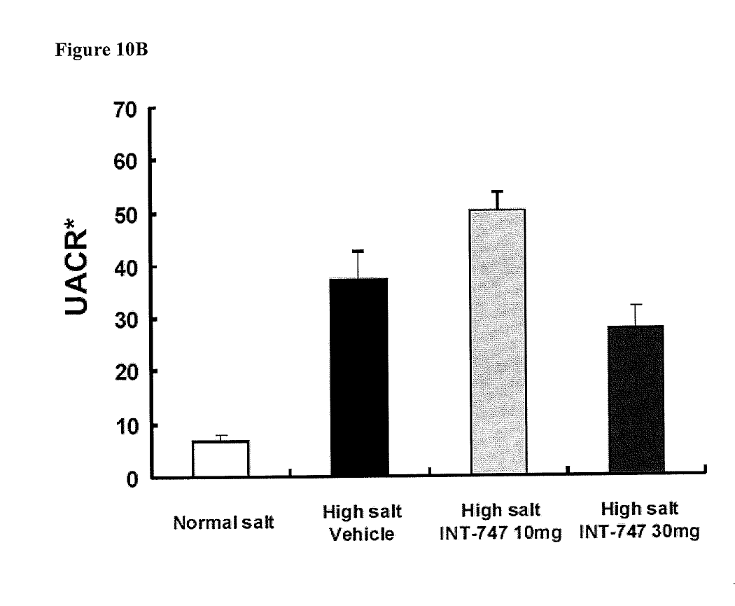

6ECDCA treatment showed renal protection at the dose of 30 mg/kg. 6ECDCA reduced albuminuria induced by high salt diet (FIGS. 10A-B).

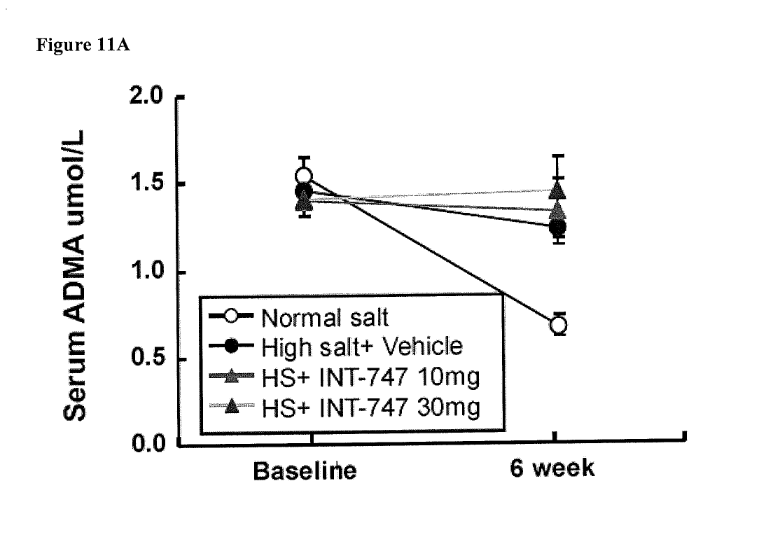

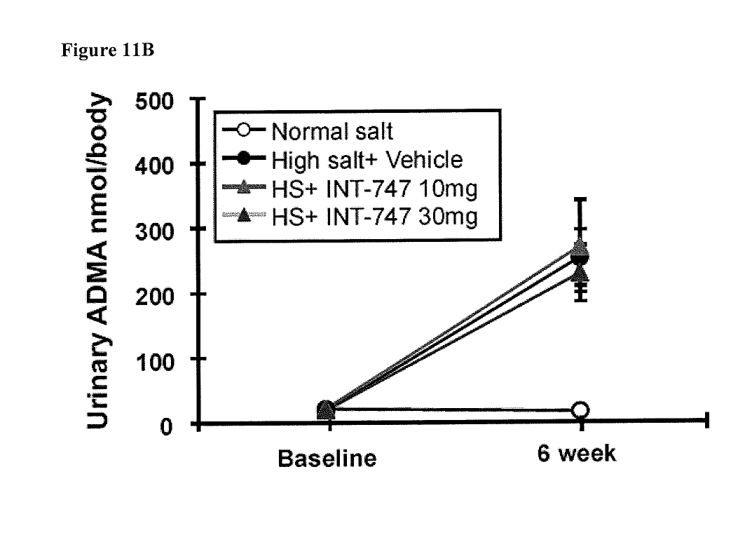

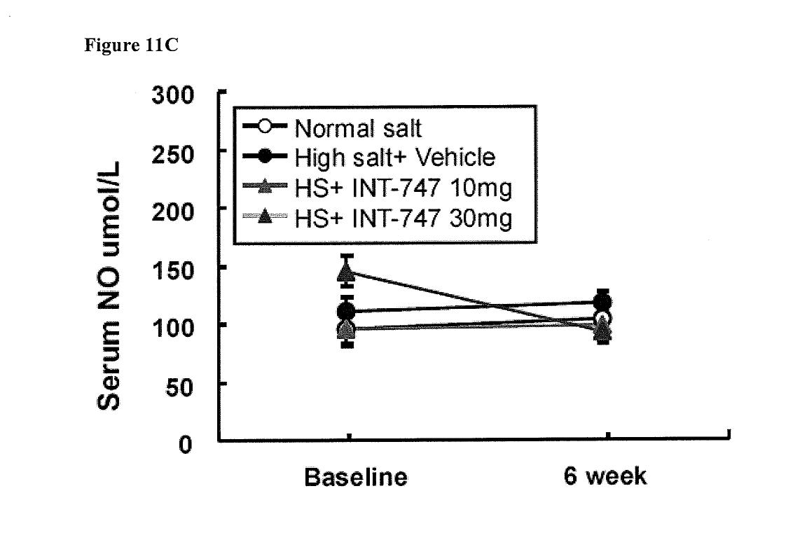

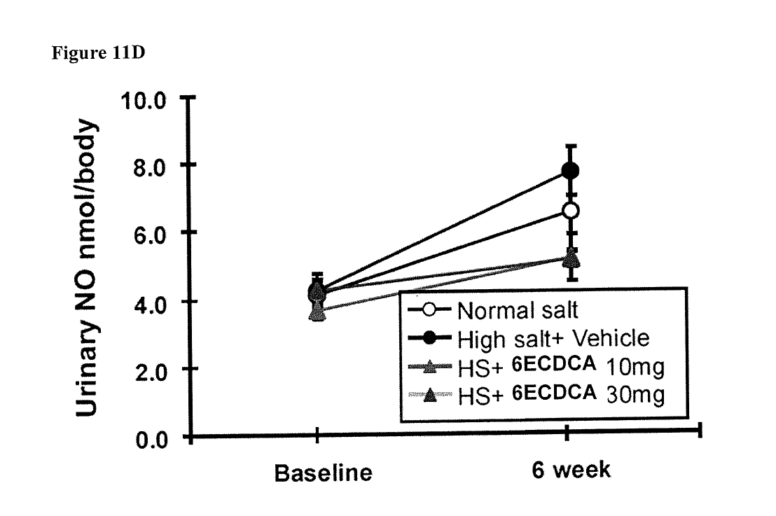

6ECDCA treatment did not reduce ADMA nor increase NO levels in serum and urine (FIG. 11).

DSS rats fed an 8% NaCl (HS) diet generally manifest an increase in blood pressure and mortality, associated and cardiac and renal hypertrophy, and lung congestion (as manifested by increased organ weights). DSS rats fed a HS diet also manifest insulin resistance. Glucose and insulin levels trended lower in the 6ECDCA treated animals, and the IR-index was reduced by 38% and 21% in the 6ECDCA 10 mg/kg and 30 mg/kg treated animals compared to the vehicle, respectively. 6ECDCA did not reduce blood pressure in DSS rats fed HS. 6ECDCA had beneficial effects on renal function (reduced albuminuria), and also reduced pulmonary congestion. These effects are independent of systemic changes in ADMA and NO levels.

### Example 5

### Effects of Chronic Treatment with the Compound 1 (Also Referred to As OCA) in the Monocrotaline-Induced Pulmonary Hypertensive Rat Model

MCT-Induced Pulmonary Hypertensive Rat Model

Pulmonary hypertension was induced by subcutaneous injection of 60 mg/kg monocrotaline (MCT) (Sigma Chemicals, St. Louis, Mo., USA) dissolved in 0.5 N HCl solution. Briefly, male Sprague-Dawley (SD) rats, weighing between 200 to 250 g, were housed in climate-controlled conditions with a 12 hours light:12 hours dark cycle, with free access to chow and water. SD rats were randomly allocated to the following groups:
1) SD rats left untreated and sacrificed after 7 (n=5) or 28 (n=5) days;
2) SD rats receiving a single subcutaneous injection of vehicle MCT sacrificed after 7 days (n=5);
3) SD rats receiving a single subcutaneous injection of vehicle MCT sacrificed after 28 days (n=10);
4) SD rats receiving a single subcutaneous injection of MCT [60 mg/kg, n=59 sacrificed after 7 days;
5) SD rats receiving single subcutaneous injection of MCT [60 mg/kg, n=159 sacrificed after 28 days;
6) SD rats receiving single subcutaneous injection of MCT [60 mg/kg] and immediately treated with OCA (30 mg/kg, daily 5 days a week, by oral gavage, n=5) for 7 days];
7) SD rats receiving single subcutaneous injection of MCT [60 mg/kg] and immediately treated with OCA (30 mg/kg, daily 5 days a week, by oral gavage, n=10) for 28 days];
8) SD rats receiving single subcutaneous injection of MCT [60 mg/kg] and immediately treated with tadalafil (10 mg/kg/day, in drinking water, n=5) for 7 days];
9) SD rats receiving single subcutaneous injection of MCT [60 mg/kg] and immediately treated with tadalafil (10 mg/kg/day, in drinking water, n=10) for 28 days];
10) SD rats receiving single subcutaneous injection of MCT [60 mg/kg] and immediately treated with vehicle OCA (by oral gavage, n=5) for 7 days;
11) SD rats receiving single subcutaneous injection of MCT [60 mg/kg] and immediately treated with vehicle OCA (by oral gavage, n=10) for 28 days.

The rats were weighed and observed for general appearance during the study period. The rats were sacrificed by cervical dislocation after 7 or 28 days and specimens of lung and heart were harvested and processed for subsequent analyses. Animal handling was complied with the Institutional Animal Care and Use Committee of the University of Florence, Florence, Italy, in accordance to the Italian Ministerial Law #116/92.

After animal sacrifice, the right ventricle (RV), left ventricle, and interventricular septum (LV+S) were weighted. The RV to LV+S ratio [RV/(LV+S)] were used as an index of right ventricular hypertrophy (RVH)

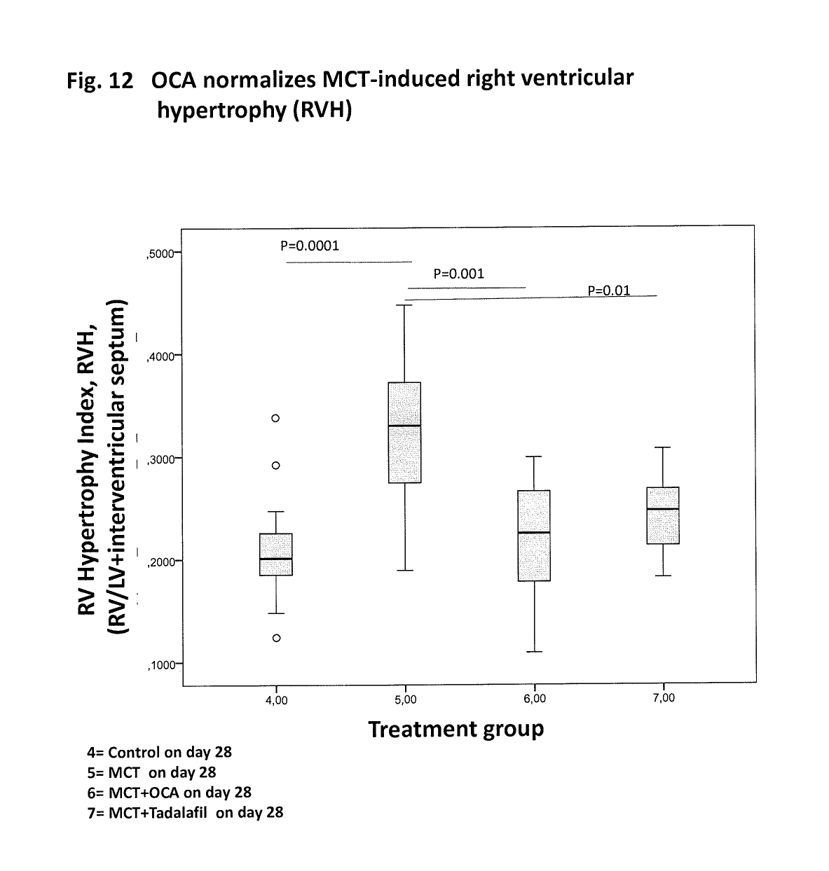

MCT induced an increase in the right ventricular hypertrophy index (RVH) at day 7 (not shown), reaching statistical significance at day 28, compared with the control group (FIG. 12). Treatment with OCA completely normalized RVH at day 28. Similar results were observed after a 28 day treatment with Tadalafil (FIG. 12).

Pulmonary vascular remodeling was evaluated by determining wall thickness (WT). Lungs were fixed in 10% buffered formalin and embedded in paraffin, then sectioned at a thickness of 5 μm and stained with hematoxylin-eosin. Ten pulmonary arteries were examined for structural integrity using a morphometric image analysis system independently by two pathologists who were blinded to animal grouping. WT, vessel diameter (ED), were determined. WT(%)=(2×WT/ED)×100%. More than 50 images of pulmonary arterioles (25 to 100 μm diameters) from at least three tissue sections at a magnification of ×20 were captured using amicroscopic digital camera and analyzed using an image analysisprogram (Fiji-win32). The external diameter (D) and medial thickness oneither side (M1 and M2) were measured along the shortest diameter. The medial wall thickness was expressed as follows: % wall thickness=[(M1+M2)/2/D]×100.

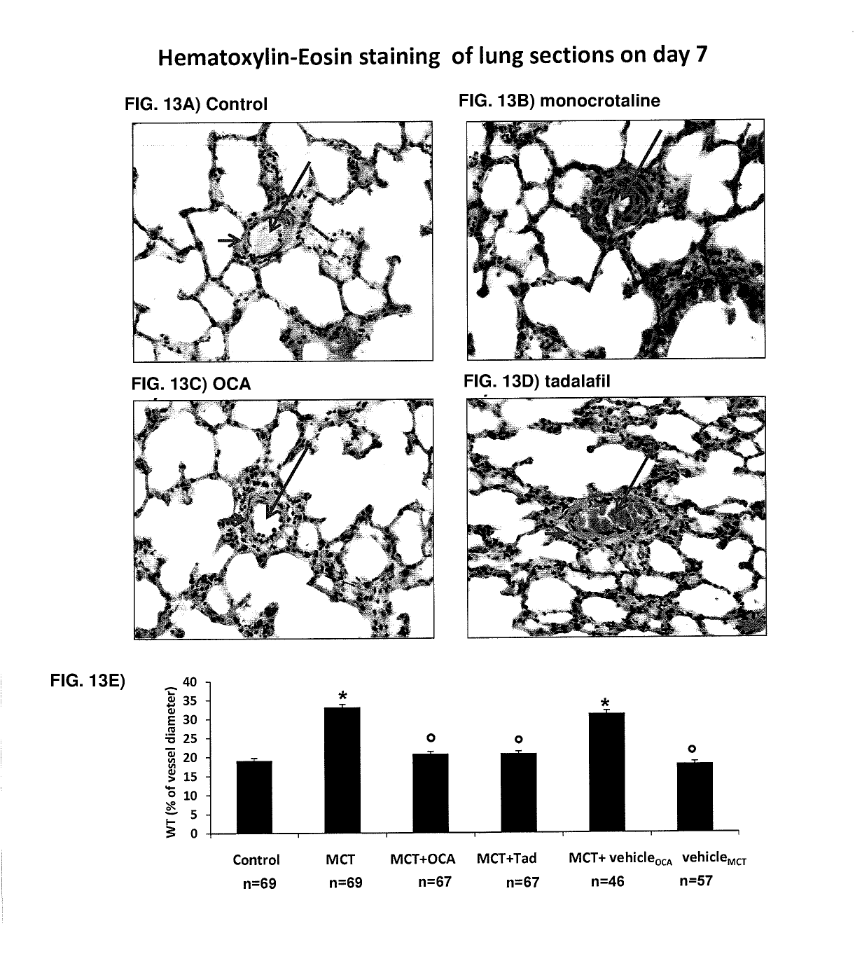

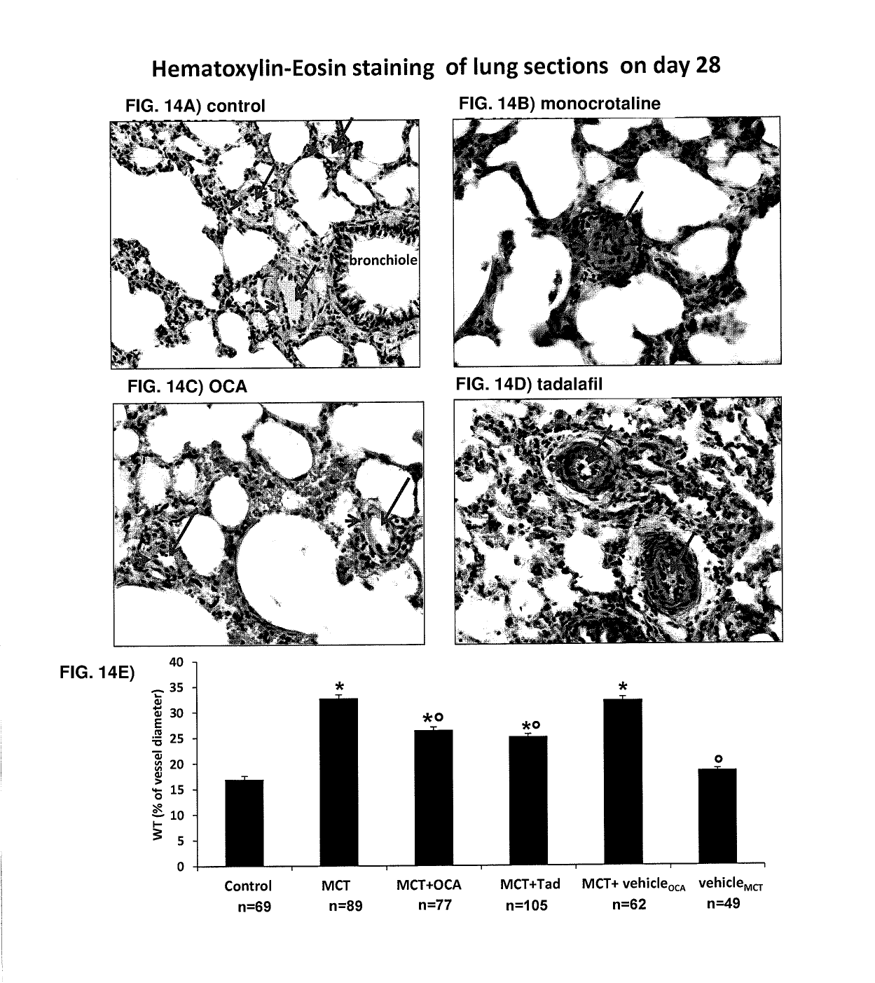

The medial wall thickness in the different experimental groups was examined (FIGS. 13, 14). MCT induced a marked increase in the wall thickness (WT) in small pulmonary arteries compared with control, either on day 7 (FIG. 13) or on day 28 (FIG. 14) [on day 7: (33±0.8% in MCT vs. 19±0.7 in control; p<0.00001); on day 28: (32.6±0.7% in MCT vs. 16.8±0.8% in control , p<0.00001)]. OCA treatment both on day 7 and day 28 markedly reduced the MCT-induced increase in WT (both p<0.00001, FIGS. 13 and 14, respectively).

mRNA expression analysis of genes involved in inflammation [interleukin 6 (IL-6), monocyte chemoattractant protein-1 (MCP-1/CCL2), cyclooxygenase-2], endothelium-proliferative factors (vascular endothelial growth factor (VEGF) and angiotensin-converting enzyme 2 (ACE2)), NO-signaling [endothelial nitric oxide synthase (eNOS), phosphodiesterase type 5 (PDE5), cyclic guanylate cyclase subunits type 1a3 and 1b3, GC1a3, GC1b3, protein kinase G 1, PKG1], were performed. Isolation of RNA from lung, qRT-PCR was performed according to the fluorescent TaqMan methodology. PCR primers and probes specific for mRNA sequences of aforementioned target genes and of the reference gene 18S rRNA was purchased from Life Technologies (Paisley, UK).

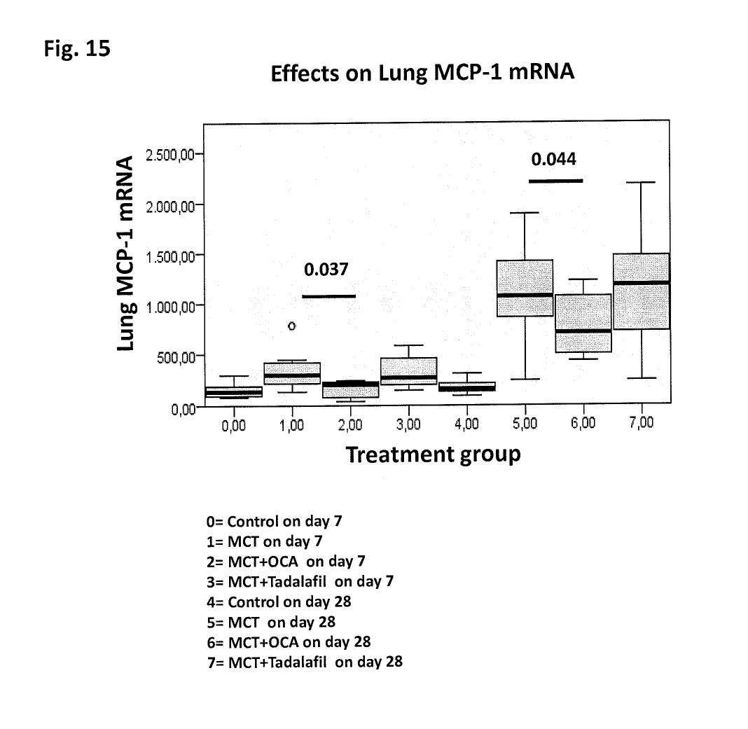

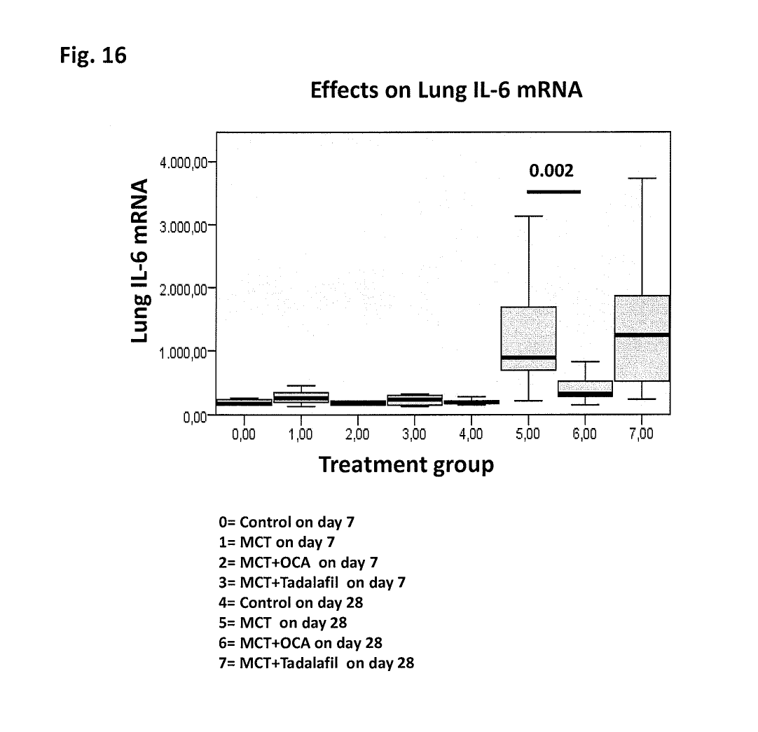

Gene expression of MCP-1 was significantly increased after MCT treatment both at day 7 and 28. Interestingly, OCA treatment significantly reduced MCT-induced MCP-1 expression both at 7 and 28 days (FIG. 15). Similarly, at day 28 OCA significantly reduced IL-6 mRNA expression, which was upregulated by MCT dosing (FIG. 16).

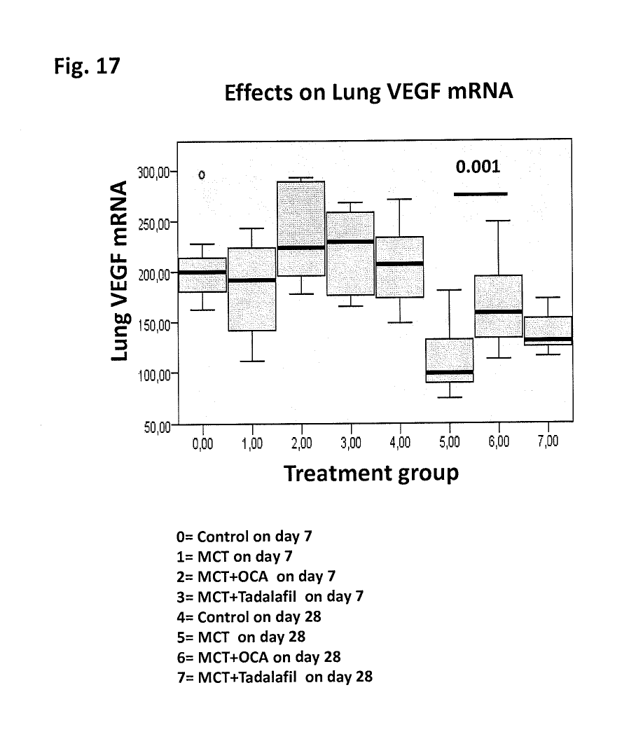

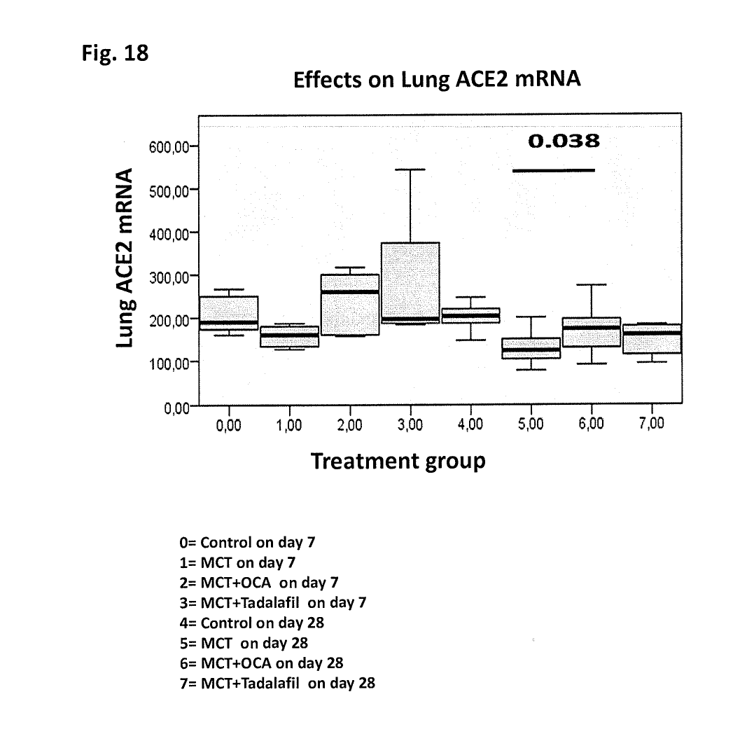

Expression of VEGF and ACE2 genes were not significantly different among groups on day 7. Conversely, both showed a decrease in the MCT group compared with the control group on day 28. OCA treatment was able to significantly upregulate both VEGF (FIG. 17) and ACE2 (FIG. 18) mRNA expression (p=0.001 and p=0.038, respectively, vs. MCT at the same time point).

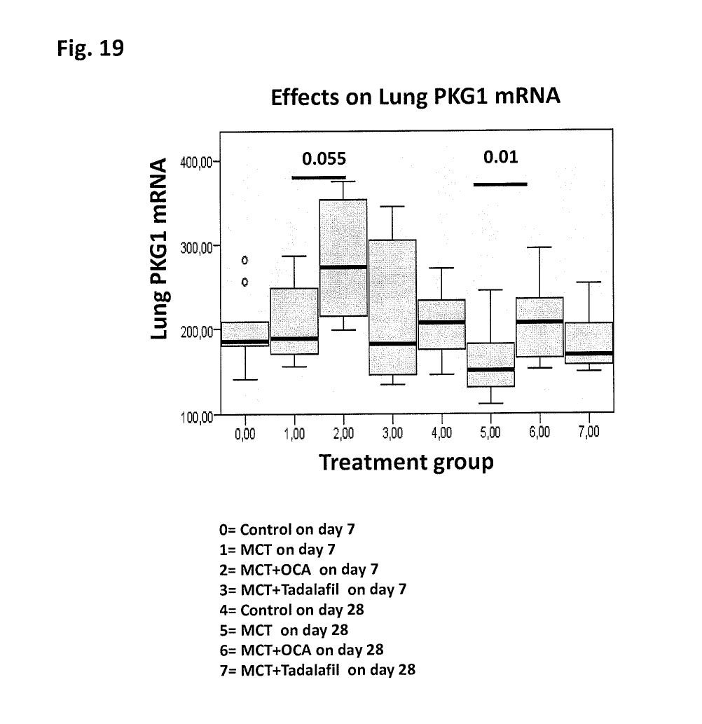

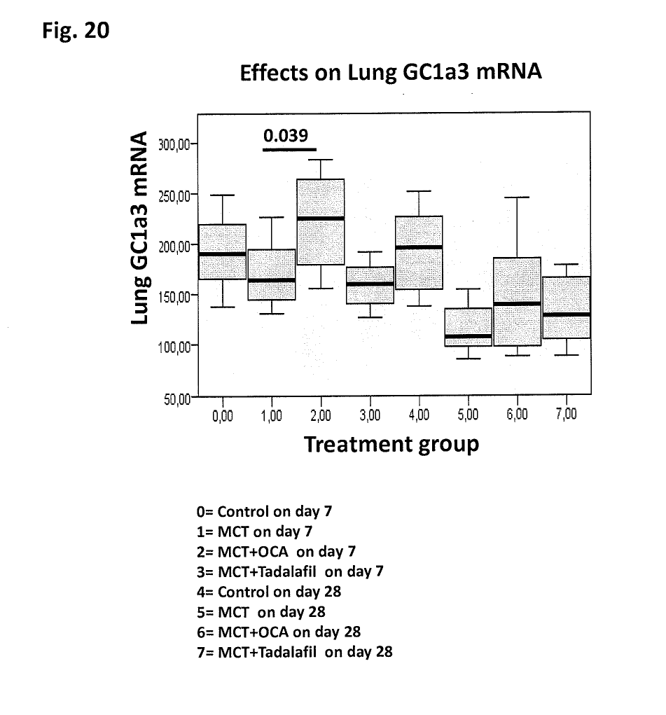

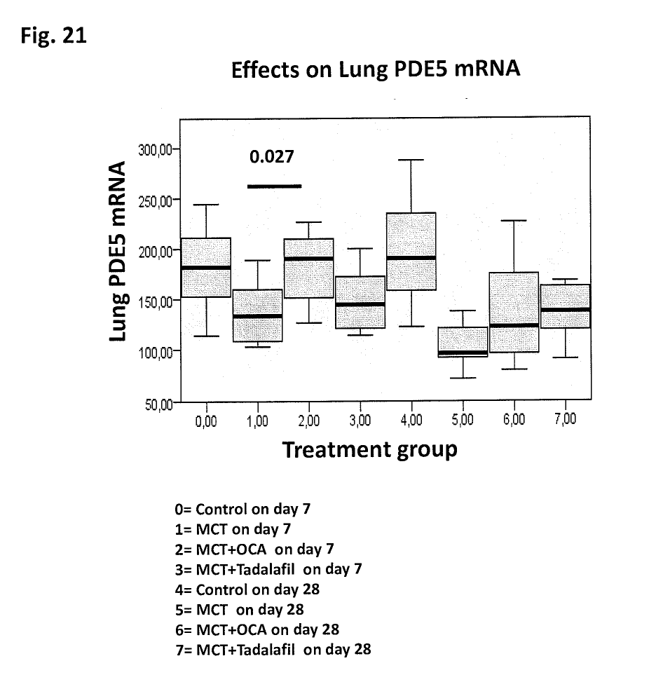

After 7 days of OCA dosing, there was a significant increase of genes related to NO-signaling, including GC1a3, PKG1 and PDE5 (FIGS. 19-21). On day 28, there was a significant reduction in the expression of all these genes (all p<0.01 vs. control at the same time-point). The 28-day treatment with OCA significantly upregulates PKG1 mRNA expression (p=0.01 vs. MCT at the same time point; FIG. 19).

During the study period, the animals were observed daily for mortality, and median survival time in each group was calculated with Kaplan-Meier analysis.

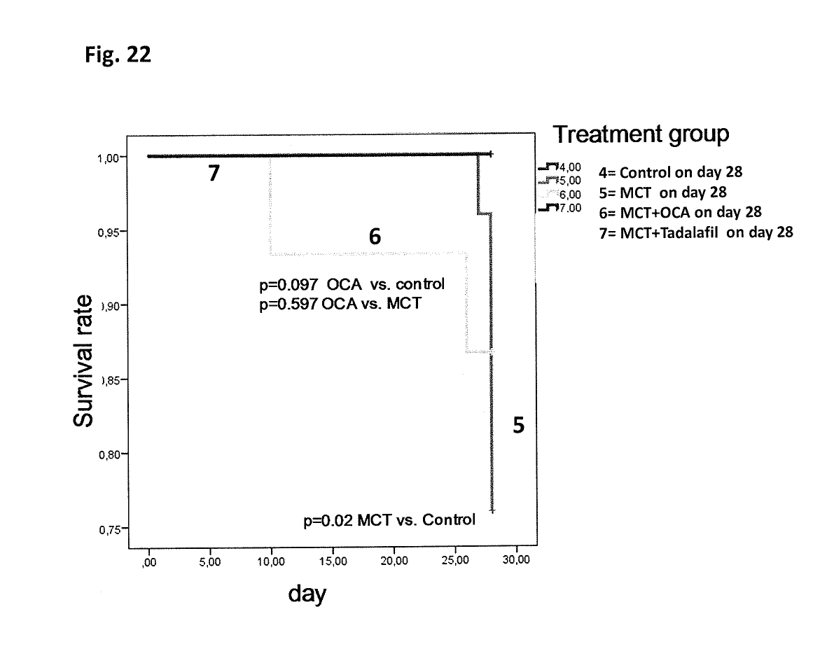

FIG. 22 shows univariate analysis of survival in untreated or MCT-treated rats. Mortality was observed daily and the survival rate was shown at each time point indicated. The survival rate on day 0 at the start of treatment was 100%. MCT induces a statistically significant decrease in survival (p=0.022). OCA decreases the number of deaths from 24 to 13.2%. Although this decrease was not statistically different from MCT, it resulted also not different from control.

Results are expressed as mean±S.E.M. (standard error of the mean) for n experiments as specified. The statistical analysis was performed with a one-way ANOVA test followed by the Tukey-Kramer post hoc analysis in order to evaluate differences between the groups, and p<0.05 was considered significant. When data were non-normally distributed, statistical differences were calculated with Kruskal-Wallis test and Mann-Whitney U-test was used for comparisons between groups. Correlations were assessed using Spearman's method and statistical analysis was performed with the Statistical Package for the Social Sciences (SPSS, Inc., Chicago, Ill., USA) for Windows 20.0.

1. A method of treating or alleviating a pulmonary disease or condition caused by or associated with narrowing of the pulmonary blood vessels in a subject, comprising administering to the subject a therapeutically effective amount of a compound of formula A: 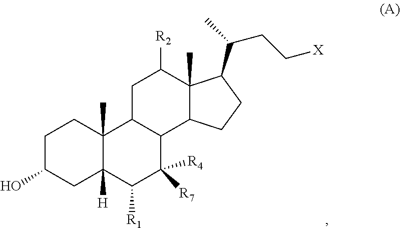
or a pharmaceutically acceptable salt thereof, wherein:
R₁ is hydrogen or unsubstituted C₁-C₆ alkyl;
R₂ is hydrogen or α-hydroxyl;
X is C(O)OH, C(O)NH(CH₂)ₘSO₃H, or C(O)NH(CH₂)ₙCO₂H;
R₄ is hydroxyl or hydrogen;
R₇ is hydroxyl or hydrogen;
m is an integer 1, 2, or 3; and
n is an integer 1, 2, or 3.

2. The method of claim 1, wherein R₁ is unsubstituted C₁-C₆ alkyl.

3. The method of claim 2, wherein R₁ is methyl, ethyl, or propyl.

4. The method of claim 3, wherein R₁ is ethyl.

5. The method of claim 1, wherein R₁ is selected from methyl, ethyl and propyl; R₄ is OH; R₇ is H; and R₂ is H.

6. The method of claim 1, wherein the compound is 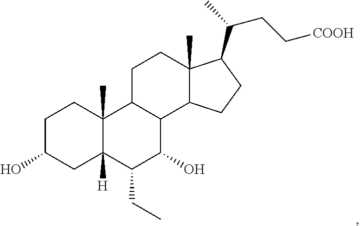 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]
or a pharmaceutically acceptable salt thereof.

7. The method of claim 1, wherein the pulmonary disease or condition is selected from obstructive pulmonary disease (COPD), emphysema, asthma, idiopathic pulmonary fibrosis, pneumonia, tuberculosis, cystic fibrosis, bronchitis, pulmonary hypertension, Primary Pulmonary Hypertension (PPH), Secondary Pulmonary Hypertension (SPH), interstitial lung disease, and lung cancer.

8. The method of claim 7, wherein the pulmonary disease or condition is selected from COPD, emphysema, asthma, cystic fibrosis, and pulmonary hypertension.

9. The method of claim 8, wherein the pulmonary disease or condition is pulmonary hypertension.

10. The method of claim 9, wherein the pulmonary hypertension is idiopathic pulmonary arterial hypertension (IPAH) or secondary pulmonary hypertension (SPH).

11. The method of claim 1, wherein the subject is a human.

12. The method of claim 1, wherein the compound is administered systemically, orally, intravenously, intramuscularly, intraperitoneally, or by inhalation.
